(19) United States
(12) Patent Application Publication
(10) Pub. No.: US 2023/0301851 A1
(43) Pub. Date: Sep. 28, 2023

(54) SEATING SYSTEM

(71) Applicant: Maker Healthcare, Inc., Wilmington, DE (US)

(72) Inventors: NATHAN P. CLEMENTE, Chicago, IL (US); THOMAS P. JENSEN, Eagle, ID (US); STEVEN D. MUSCHLER, Chicago, IL (US); RYAN RAMOS, Milwaukee, WI (US); PERRY SELLERS, Hudsonville, MI (US); JASON PITTMAN, Waukesha, WI (US)

(73) Assignee: Maker Healthcare, Inc., Wilmington, DE (US)

(21) Appl. No.: 18/161,361

(22) Filed: Jan. 30, 2023

Related U.S. Application Data
(63) Continuation of application No. PCT/US2023/061518, filed on Jan. 27, 2023.
(60) Provisional application No. 63/304,534, filed on Jan. 28, 2022.

(51) Int. Cl. A61G 5/10; A61G 5/12

(52) U.S. Cl. CPC A61G 5/1067; A61G 5/1013; A61G 5/1043; A61G 5/1064; A61G 5/127; A61G 5/128; A61G 2203/10

(57) ABSTRACT

A seating system including a seat portion having a seating surface and a back portion having a support surface is disclosed. The seating system may be configured in a seated (or upright) configuration in which the seating surface of the seat portion is generally perpendicular to the support surface of the back portion. The seating system may also be configured in a stored configuration in which the seating surface of the seat portion is generally parallel with and faces towards the support surface of the back portion. The seating system may also be configured in a recumbent position in which the seating surface of the seat portion is generally parallel and faces in the same direction as the support surface of the back portion.

### CROSS REFERENCE TO RELATED APPLICATIONS

This application is a Continuation of International Patent Application Serial No. PCT/US23/61518 filed on Jan. 27, 2023, the entire disclosure of which is incorporated by reference herein for all purposes. This application is also based on and claims priority to U.S. Provisional Application Serial No. 63/304,534 filed Jan. 28, 2022, the entire disclosure of which is hereby incorporated by reference herein for all purposes.

### BACKGROUND OF THE INVENTION

Mobile seating systems such as wheelchairs are widely used to provide users with medical conditions the ability to move about. But wheelchairs have not typically been designed with the user in mind. Instead, traditional wheelchairs may be unintelligent, uncomfortable, unwieldy, and unhelpful to a user, a caregiver, a medical provider, and others. For example, some wheelchairs provide little more than a piece of fabric for a seat and back rest, providing little support to the user. In contrast, some wheelchairs provide rigid materials for a seat and back rest, causing discomfort to the user. Some wheelchairs may also be difficult to transport. For example, many electronic wheelchairs include bulky motors that preclude traditional transportation options. And wheelchairs typically provide little functional utility other than mobility. For example, when a medical provider wants to run medical tests on the user, the user must be removed from the wheelchair and placed on a separate surface (such as a gurney or exam table). And wheelchairs with additional functionality are heavy and cumbersome to operate.

In addition, wheelchairs can cause more medical problems for a user. For example, sitting in a wheelchair can increase the risk of developing bed sores, skin tears, and infections. As another example, sitting in a wheelchair can exacerbate pain for users with hip, back, and leg problems. Existing wheelchairs do not address user needs.

### SUMMARY OF THE INVENTION

The disclosed subject matter includes a system such as a mobility system, a mobile seating device, or a human transport device. The mobility system may be a system that assists the user in moving by themselves (i.e., without the assistance of another person), such as a wheelchair, an electric scooter, a rollator, or a walker. A mobile seating device may be any device that provides mobility while a user is seated such as a wheelchair or an electric scooter. A human transport device (e.g., a patient transport device) may be any device that assists with transporting a human. For example, the human transport device may be a wheelchair or a gurney designed to transport patients with medical conditions. Although the embodiments described herein will use the example of a wheelchair, the disclosed subject matter may be used with other mobility systems, mobile seating devices, and/or human transport devices. In some examples, the disclosed subject matter may also include stationary seating systems. For purposes of explanation, the phrase “seating system” will be used to collectively refer to mobility systems, mobile seating devices, human transport devices, and stationary seating systems.

In at least one embodiment, the disclosed seating systems may include a seat portion having a seating surface and a back portion having a support surface. In a seating (or upright) configuration, the seating surface of the seat portion may be generally perpendicular to the support surface of the back portion such that the seating surface of the seat portion faces in a first direction and the support surface of the back portion faces in a second direction generally perpendicular to the first direction. This allows the seating system to function as a chair and allows the user to sit in the seating system. In a stored configuration, the seating surface of the seat portion may be generally parallel to the support surface of the back portion, and the seating surface of the seat portion may face an opposite direction as the support surface faces such that the seating surface of the seat portion faces the support surface of the back portion. For example, the support surface of the back portion may be located above the seating surface of the seat portion, the support surface of the back portion may face downwards, and the seating surface of the seat portion may face upwards. This allows the seating system to be collapsed for storage or transportation. The back portion may be rotatably connected to the frame of the seating system or to the seat portion to allow it to rotate relative to the seat portion. The seat portion may be detachably connected to the frame on a first lateral side and rotatably connected to the frame on a second lateral side opposite the first lateral side, which may allow both the seat portion and the back portion to rotate to a position generally perpendicular to the position of the seat portion in the seating (or upright) configuration.

In at least one embodiment, the disclosed seating systems may include first and second primary wheels connected by an extendable axle. The axle may be configured in an elongated configuration when the seating system is in the seating configuration. The axle may be configured in a shortened configuration when the seating system is in the stored configuration. When the axle is in the shortened configuration, the first and second primary wheels may be generally parallel with the seating surface of the seat portion and the support surface of the back portion, and the seat portion and back portion may be located between the first primary wheel and the second primary wheel. In other embodiments, when the axle is in the shortened configuration, the seat portion and back portion may be generally perpendicular to the first and second primary wheels and may be located outside of the area between the first and second primary wheels.

In at least one embodiment, the disclosed seating systems may also include one or more adjustable support rods connected to footpads. The support rods may be lengthened or shortened depending on the needs of the user. The support rods may be rotatably connected to the frame. In the seating configuration, the adjustable support rods may extend downward from the seat portion. In the stored configuration, the adjustable support rods may extend generally parallel with and towards the seat portion such that they are underneath the seat portion, or towards the back of the seating system if the seat portion has been rotated from its usual position in the seated configuration. The footpads may be rotatably connected to the support rods and may rotate between a first position generally perpendicular to the support rods and a second position generally parallel with the support rods. Calf pads may also be rotatably connected to the adjustable support rods and may be rotatable between a first position and a second position generally perpendicular to the first position. This may allow users to be provided with support for their calves during use (when the calf pads are in the first position) and then allow the calf pads to be rotated to the second position such that the calf pads do not impede the user from reaching their legs to the floor and standing up. The calf pads may also be rotatable to a third position generally parallel with the second position on the opposite side of the support rods for storage.

In at least one embodiment, in a recumbent configuration, the disclosed seating systems may be positioned with the seat portion generally parallel with the back portion, and with the seating surface of the seat portion facing in the same direction that the support surface of the back portion faces. The adjustable support rods may be positioned parallel with and extending generally away from the seat portion. The user may use the seating system as a gurney or the like when in this position.

In at least one embodiment, the disclosed seating systems may have a braking mechanism including a brake connected to a brake control. The brake may be a circular brake (such as a disc brake) located laterally outside a rim of a first primary wheel, that is, on the side of the primary wheel opposite the seat portion and the other primary wheel. The brake control may be located radially outside of the first primary wheel and the second primary wheel. The brake control may be disposed on the frame on a same side as the first primary wheel and located at a distance from the first primary wheel. The distance between the brake control and the rim of the primary wheel may be adjustable. A locking mechanism may be used to maintain the adjustable distance during use. The brake control may be connected to the brake (e.g., through a hydraulic or pneumatic connection) and may cause the brake to activate (to slow or stop the primary wheel) when the brake control is selected. The brake control may be selected by pushing or pulling the brake control towards the frame.

In at least one embodiment, the disclosed seating systems may have a seat portion that is slidable. In some embodiments, the seat portion may be slidably connected to the frame. In other examples, a first portion of the seat portion may be slidably connected to a second portion of the seat portion. The user may slide the seat portion towards and away from the back portion. This allows the user to adjust the location of the seat relative to the back portion to enhance user comfort. In some embodiments, the seat portion may additionally or alternatively be configured to slide in a lateral direction, e.g., to assist the user in entering and exiting the seating system.

In at least one embodiment, the disclosed seating systems may have armrests located on both sides of the seat portion. The armrests may be rotatably connected to the frame. In some examples, the armrests may be rotatably connected to an axle that connects the first primary wheel to the second primary wheel. In other examples, the armrests may be rotatable between a first position in which the armrest extends forward, generally parallel with a first primary wheel, and generally perpendicular to the back portion, and a second position in which the armrest extends laterally outward, generally perpendicular to the first primary wheel, and generally parallel with the back portion. The armrests may be rotated out of the way to allow the user to enter or exit the seating system. The armrests may also include indicator lights, a display screen, control buttons, a joystick, and/or sensors such as a pulse oximeter, a heart rate sensor, a temperature sensor, or other biometric sensors. In some examples, the armrests may include a removable portion that can be replaced with other removable portions such that the user can select a suitable removable portion for a particular activity. For example, the removable portions may include a removable support armrest, a removable tray, a removable cupholder, or a removable stand.

In at least one embodiment, the disclosed seating systems may include a push bar. The push bar may be rotatably connected to seating system (e.g., to the frame or an axle connecting the first and second primary wheels) and may be rotated out of the way when the back portion is rotated into the recumbent configuration and/or for storage.

In at least one embodiment, the disclosed seating system may have lights located on the footpads, on the back of the seating system, on primary or secondary wheels (e.g., on the hubs, the rim, or the spokes), on the push bar, or in other useful locations. The disclosed seating systems may also include one or more reflectors, which may be located on the rear-facing or lateral portions of the seating system.

In at least one embodiment, the disclosed seating systems may have an inflatable cushion on (e.g., in, attached to, or located atop) the seating surface. The inflatable cushion may have two or more inflatable chambers each connected to a fluid source through an intake valve. An exhaust valve may be used to decrease the pressure in the inflatable chamber. The fluid source may be, for example, a pressure tank or a compressor. The intake and exhaust valves may be controlled by one or more control circuits, which may be configured to adjust the pressure in each inflatable cushion according to pre-defined settings, user controls, or other criteria. The valves may include a poppet including a first surface perpendicular to the direction of fluid flow, and second and third surfaces angled relative to the first surface and configured to engage with first and second surfaces of a valve seat when the valve is closed. An actuator connected to the poppet by a valve stem and controlled by the control circuits may be used to adjust the location of the poppet and thereby open or close the valve.

In at least one embodiment, the disclosed seating systems may have an inflatable cushion including one or more inflatable chambers. Each of the inflatable chambers may be integrally formed with one or more valves, such as an intake valve and/or an exhaust valve. For example, the inflatable chamber may be a 3-D printed bladder including an intake valve, an exhaust valve, or a combination intake valve and exhaust valve.

Seating systems in accordance with the disclosed subject matter may include one or more integrated sensors, including without limitation one or more pressure sensors, one or more temperature sensors, one or more heart rate sensors, and one or more moisture sensors. By integrating intelligent sensors that can read data within a modular ecosystem clinicians, caregivers, and family members can not only monitor their activities but can prescribe treatments that may lead to better health outcomes. This ecosystem of synthesized data may be sent to an electronic medical record system to assist clinicians in their treatment plans.

The disclosed subject matter also includes systems including the disclosed mobility systems, mobile seating devices, human transport devices, and stationary seating systems and a backend system. The backend system may be configured to store information received from the disclosed seating systems, such as biometric sensor data. The backend system may also interface with other systems such as an electronic medical records system, a web portal, or a software application on a medical device to provide biometric sensor data to a user’s medical provider, caregiver, or friends and family.

The disclosed subject matter also includes methods performed by, using, or related to the disclosed mobility systems, mobile seating devices, human transport devices, and/or stationary seating systems. Such methods may include methods for adjusting the pressure of a fluid-filled cushion automatically or based on user feedback, methods for detecting an alert condition, methods for activating an alert based on detecting an alert condition, methods for providing sensor data to a backend system and/or an electronic medical records system, methods for developing a treatment plan for a user based on sensor data received by an electronic medical records system, and the like.

The disclosed mobility systems, mobile seating devices, human transport devices, and/or stationary seating systems may be used by a user who may have health issues. The user may have a caregiver that provides assistance to the user. In some examples, the caregiver may be a trained medical professional such as a hired nurse. In some other examples, the caregiver may be a family member or other person that does not have medical training. The user may also have a medical or other care provider (e.g., a primary care physician or another doctor or nurse, or a physical or occupational therapist). The medical provider may be different than the caregiver. In some embodiments, the disclosed system may provide medical providers and/or caregivers the ability to monitor the user and his or her health.

### BRIEF DESCRIPTION OF THE DRAWINGS

FIG. 1 is a perspective view of a first embodiment of a human transport system in accordance with the disclosed subject matter.

FIG. 2 is a perspective view of a second embodiment of a human transport system in accordance with the disclosed subject matter.

FIG. 3 is a perspective view of a third embodiment of a human transport system in accordance with the disclosed subject matter.

FIG. 4 is a block diagram of a circuit board for a seating system in accordance with embodiments of the disclosed subject matter.

FIG. 5 is a flowchart describing a method for adjusting the pressure of a cushion on a seating system in accordance with embodiments of the disclosed subject matter.

FIG. 6 is a flowchart describing a method for adjusting the pressure of a cushion of a seating system in accordance with the disclosed subject matter.

FIG. 7 is a state diagram showing the states associated with adjusting the pressure of a cushion on a seating system in accordance with the disclosed subject matter.

FIG. 8 is a flowchart describing a method for activating an alert in accordance with embodiments of the disclosed subject matter.

FIG. 9 is a block diagram of a networked system in accordance with embodiments of the disclosed subject matter.

FIG. 10 is a flowchart describing a method for setting up a seating system in accordance with embodiments of the disclosed subject matter.

FIGS. 11A and 11B show screenshots of an interface for a software application in accordance with embodiments of the disclosed subject matter.

FIG. 12 is a flowchart describing a method for communicating with a medical provider computer system in accordance with embodiments of the disclosed subject matter.

FIG. 13 is a block diagram of one embodiment of the surface of an armrest of a seating system in accordance with embodiments of the disclosed subject matter.

FIG. 14 is a diagram of one embodiment of a cushion that may be used with a seating system in accordance with embodiments of the disclosed subject matter.

FIG. 15A illustrates an embodiment of a seating system in an upright or seated configuration in accordance with embodiments of the disclosed subject matter.

FIG. 15B illustrates an embodiment of a seating system with the back portion rotated relative to the configuration shown in FIG. 15A in accordance with embodiments of a disclosed method for collapsing a seating system.

FIG. 15C illustrates an embodiment of a seating system with the seat portion, back portion, and footpads rotated relative to the configuration shown in FIG. 15B in accordance with embodiments of a disclosed method for collapsing a seating system.

FIG. 15D illustrates and embodiment of a seating system with the footpads and calf pads rotated and the distance between the primary wheels decreased relative to the configuration shown in FIG. 15C in accordance with embodiments of a disclosed method for collapsing a seating system.

FIG. 16A illustrates an embodiment of a seating system in an upright or seated configuration in accordance with embodiments of the disclosed subject matter.

FIG. 16B illustrates an embodiment of a seating system showing how a push bar may be unlocked and rotated backwards in accordance with embodiments of the disclosed subject matter.

FIG. 16C illustrates an embodiment of a seating system showing how a back portion may be rotated to a reclined or recumbent position in accordance with embodiments of the disclosed subject matter.

FIG. 16D illustrates an embodiment of a seating system showing how both the push bar and the back portion may be rotated in accordance with embodiments of the disclosed subject matter.

FIG. 17 illustrates a method for sliding a cushion of a human transport system in accordance with embodiments of the disclosed subject matter.

FIG. 18 is a top view of a human transport system according to the embodiment depicted in FIG. 1.

FIG. 19 is a back view of a human transport system according to the embodiment depicted in FIG. 1.

FIG. 20 is a front view of a human transport system according to the embodiment depicted in FIG. 1.

FIG. 21 is a side view of a human transport system according to the embodiment depicted in FIG. 1.

FIG. 22 illustrates an embodiment of a valve that may be used in connection with inflatable cushions in accordance with embodiments of the disclosed subject matter.

FIG. 23 illustrates a second embodiment of a valve that may be used in connection with inflatable cushions in accordance with embodiments of the disclosed subject matter.

FIG. 24 illustrates an inflatable cushion arrangement that may be used in accordance with embodiments of the disclosed subject matter.

FIG. 25 illustrates a second inflatable cushion arrangement that may be used in accordance with embodiments of the disclosed subject matter.

FIG. 26 shows an embodiment of a human transport system including cupholders and a headrest in accordance with the disclosed subject matter.

FIG. 27 shows an embodiment of a human transport system in a collapsed configuration in accordance with the disclosed subject matter.

FIG. 28 shows an embodiment of a human transport system including a removable armrest portion in accordance with the disclosed subj ect matter.

FIG. 29 shows an embodiment of a human transport system including a rotatable support rod in accordance with the disclosed subject matter.

FIG. 30 shows an embodiment of a human transport system including a rotatable armrest in accordance with the disclosed subject matter.

### DETAILED DESCRIPTION

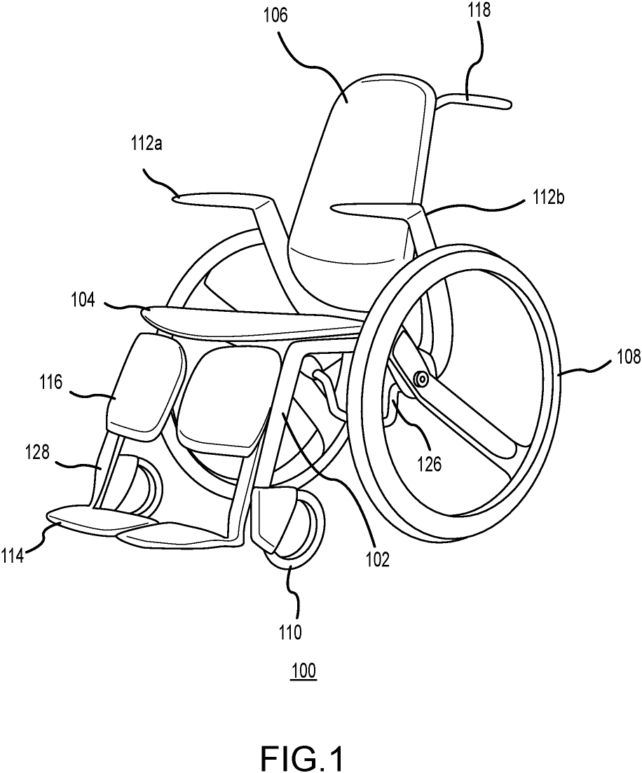

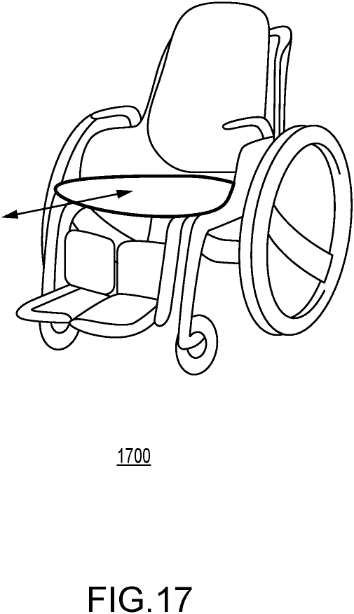

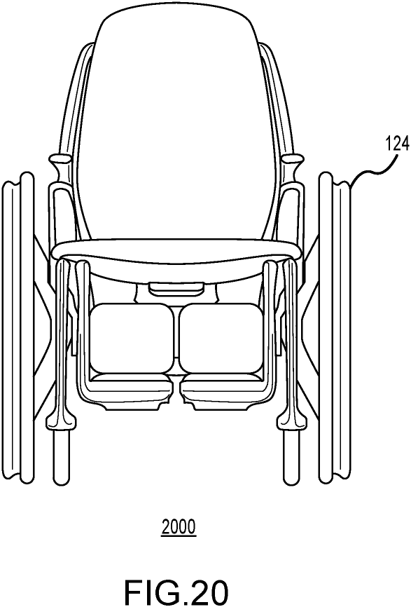

FIG. 1 shows a seating system in accordance with some embodiments of the disclosed subject matter. The wheelchair 100 may include a wheelchair frame 102 with modular components adjustably and/or removably attached thereto, which may allow the components to be easily replaced when they are worn or damaged. For example, the wheelchair 100 may include a seat portion 104, a back portion 106, primary wheels 108, secondary wheels 110, armrests 112, footpads 114, and leg pads 116. Additional views of the wheelchair 100 are shown in FIG. 18 (top view), 19 (back view), 20 (front view), and 21 (side view). The other side view is a mirror image of the side view 2100 shown in FIG. 21. However, embodiments of the disclosed subject matter may include different features on opposite sides, including cupholders, removable armrests, and other features as described and/or illustrated herein.

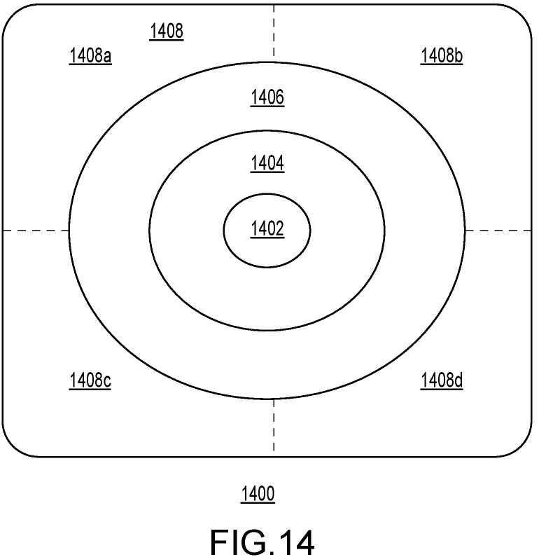

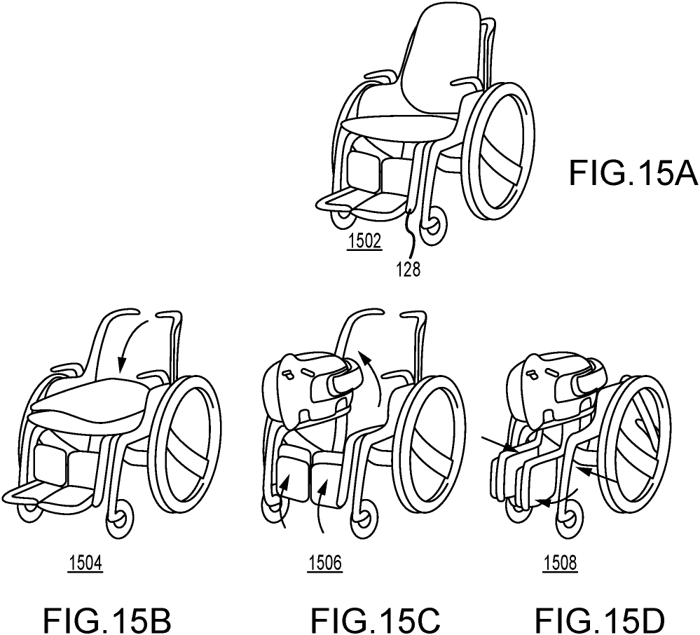

The seating system 100 may be configurable in a number of positions, including a seated position as shown in FIG. 1 which corresponds to the position of the seating system 100 during normal operation. The seated position may be referred to as the upright position as shown in FIG. 15A. As discussed below, the seating system 100 may also be configured in other positions through adjustment of the various components thereof, for example as illustrated in FIGS. 16B, 16C, and 16D, including a recumbent position in which the seating system 100 may serve as a therapy table or gurney.

The frame 102 may also be referred to as a support frame or a chassis. The frame 102 may include a first frame portion located on a first lateral side of the seating system 100 and a second frame portion located on a second lateral side of the seating system 100 opposite the first lateral side, as shown in FIG. 1. The first frame portion and second frame portion may be connected by an axle such as the extendable axle 126 described herein. The first frame portion and the second frame portion may also be connected by the seat portion 104. In some embodiments, the first frame portion and second frame portion may be additionally or alternatively connected by support elements such as bars, which may be located in front of, underneath, or behind the seat portion 104.

A seat portion 104 of a seating system 100 may include a seating surface, which refers to the surface on which the user sits during normal operation, and a back surface opposite the seating surface that faces downward during normal operation. A back portion 106 of the seating system 100 may include a support surface, which refers to the surface providing the back of the seating for the user during normal operation, and a back surface opposite the support surface that faces backwards (e.g., away from the seat portion 104, the secondary wheels, and other components located toward the front of the seating system 100) during normal operation. The angles of both the seat portion 104 and the back portion 106 may be adjustable for user comfort. For example, while some users may prefer to sit on a flat seat, other users may wish to have either the front or the back of the seat portion 104 slightly inclined. Such an incline may also assist the user in getting into or out of the seating system 100.

The seat portion 104 of the seating system 100 may be adjustably connected to the frame 102 to provide a seating surface for the user when the seating system 100 is in the seated position. In such position, the seat portion is generally parallel to the ground and is generally perpendicular to the back portion 106. As used herein, “generally perpendicular” refers to an arrangement in which the angle between two objects (here, the back portion 106 and the seat portion 104) is within 15 degrees of perpendicular in either direction. In other examples, objects referred to as “generally perpendicular” may be preferably within 10 degrees or perpendicular, more preferably within 5 degrees of perpendicular or most preferably within 2 degrees of perpendicular. The term “generally parallel” is similarly used to refer to an arrangement in which the angle between two objects is within 15 degrees of parallel in either direction but may also include angles preferably within 10 degrees of parallel, more preferably within 5 degrees of parallel, or most preferably within 2 degrees of parallel. The terms “generally perpendicular” and “generally parallel” also encompass arrangements in which the angles are perpendicular and parallel, respectively.

The seat portion 104 of the seating system 100 may be rotatably attached to the frame 102, which may assist the seating system in being folded for storage or transportation (e.g., in the trunk of a car). For example, the seat portion 104 may be rotatably attached to the frame 102 on a first lateral side of the seating system 100 (e.g., the side proximate armrest 112a) and may be detachably attached to the frame 102 on the second lateral side of the seating system 100, i.e., the lateral side opposite the first lateral side (which may correspond to the side proximate armrest 112b). In some embodiments, the seat portion 104 may be attached to the frame 102 by one or more hinges, rotary joints, pivot joints, ball joints, or other rotatable connectors. A locking mechanism may hold the seat portion 104 in place on the second lateral side when in a locked position (e.g., during operation) and allow the seat portion 104 to be detached from the second lateral side when in an unlocked position (e.g., when the user has exited the seating system 100). In some embodiments, the seat portion 104 may include at least one support bar extending laterally on the back surface of the seat portion 104. The at least one support bar may be rotatably attached to the frame 102 on the first lateral side and may be detachably attached to the frame 102 on the second lateral side to allow the seat portion 104 to rotate relative to the frame 102.

The seat portion 104 may be detached from the second lateral side of the wheelchair and rotated about the attachment on the first lateral side of the seating system 100. A depiction of the seat after it has been detached from the second lateral side of the wheelchair and rotated about the attachment on the first lateral side of the wheelchair is shown in FIG. 15C. In this manner, the seat portion 104 may be rotated from a first position (e.g., the position corresponding to the seated position as shown in FIG. 15A) to a second position generally perpendicular to the first position, allowing the seating system 100 to be collapsed when not being used by a user. This process is illustrated in FIGS. 15A through 15D. The seat portion may be rotated from the first position (the seat position as shown in FIGS. 15A and 15B) to the second position (the seat position as shown in FIGS. 15C and 15D). In this second position, the seat portion 104 and the back portion 106 may be generally parallel with the planes of the primary wheels 108. The wheelchair may then be collapsed as shown in FIG. 15D by decreasing the distance between the primary wheels 108. The seat portion 104 may be rotated in either axial direction. For example, the seat portion 104 may be rotated in a downward axial direction such that the bottom surface of the seat portion 104 faces the primary wheel 108 on the first lateral side of the seating system 100 when the seat portion 104 is in the second position. In other examples, the seat portion 104 may be rotated in an upward axial direction as shown in FIG. 15C.

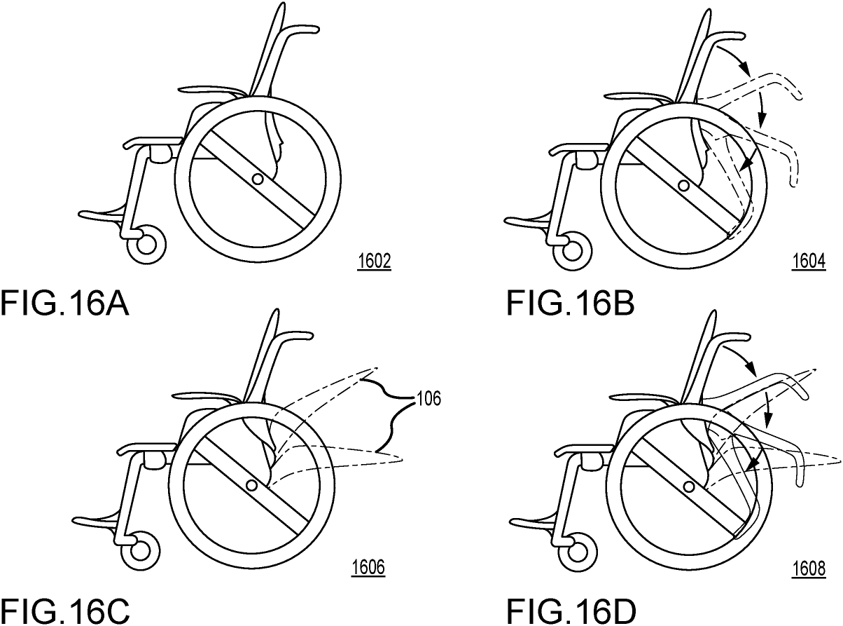

In some examples, the seat portion 104 may be slidably attached to the frame 102, e.g., on a bottom surface of the seat portion 104 opposite the seating surface. The seat portion 104 may be configured to slide forward (i.e., away from the support surface of the back portion 106) or backward (i.e., toward the support surface of the back portion 106). This configuration allows the user or a caregiver to adjust the seat portion 104 relative to the back portion 106 to account for the user’s anatomy and to decrease any discomfort the user may experience when in a particular position. The slidable configuration also allows the seat portion 104 to be moved forward to allow the user to be more easily transferred into and out of the seating system 100. A locking mechanism may prevent the seating system 104 from sliding when in a locked position (e.g., when the seating system 100 is moving) and allow the seat portion 104 to slide back and forth when in an unlocked position (e.g., to assist with the transfer of a user into or out of the seating system 100). The seating system 1700 of FIG. 17 shows a seat portion 104 that is slidably connected to the frame 102.

In some examples, the seat portion 104 may be detached from the sliding attachment with the frame 102 such that the seat portion 104 may be rotated into the second position. In an alternative arrangement, the seat portion 104 may include a first seat portion including the seating surface and a second seat portion including the bottom surface. The first seat portion may be slidably attached to the second seat portion, allowing the seating surface of the seat portion to be adjusted while avoiding the need for an additional detachable attachment and associated locking mechanism.

In some examples, the seat portion 104 could also or alternatively be slidably attached to the frame 102 such that the seat portion 104 is capable of sliding toward the first lateral side or the second lateral side of seating system 100. This configuration would allow the user to be transferred into or out of the seating system 100 from the side, which may be advantageous in certain situations (e.g., transfer to a surface that can be placed directly adjacent the lateral side of the seating system 100). As discussed below, the armrests 110 may be configured to rotated or otherwise move out of the way to enable such transfer.

The seat portion 104 may be constructed of any suitable material. For example, the seat portion 104 (as well as other components of wheelchair 100) may be constructed from fiber- or glass-filled plastics, carbon fiber injected parts, gas-assisted structural components, multi-durometer 3D printed parts, woven plastic fabric upholstery, co-molded injection materials, injection-molded nylons, or hybrid medical grade plastics such as kydex or polycarbonate.

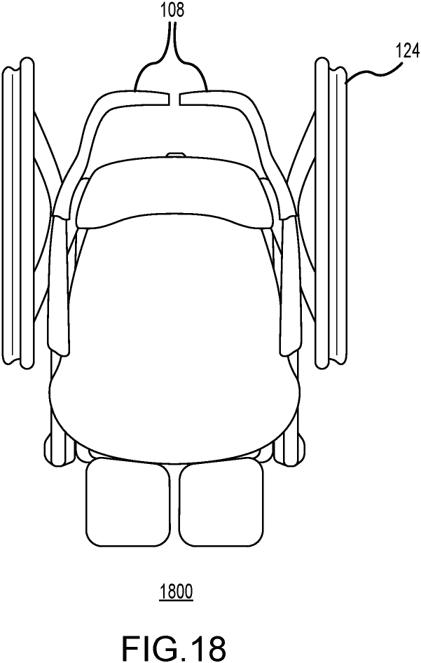

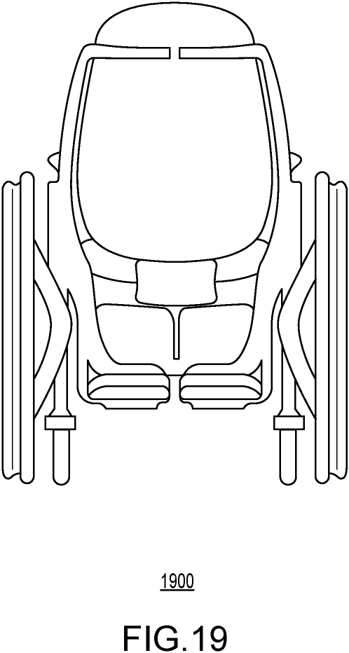

The back portion 106 may be adjustably connected to the seat portion 104, as shown for example in FIGS. 15C and 19, or to the frame 102 to provide a support surface for the user to lean against when the seating system 100 is in the seated position. In such a position, the back portion 106 is generally perpendicular to the ground and generally perpendicular to the seat portion 104. For example, both the support surface of the back portion 106 and the seating surface of the seat portion 104 may be facing upwards. The back portion 106 may be connected to the frame 102 along a lower edge of the back portion 106. For example, the back portion 106 may be rotatably attached to an axle of the seating system 100 which connects the central hubs of the primary wheels 108. In some other examples, the back portion 106 could be rotatably attached a portion of the frame 102 that is extends parallel to and is at generally the same height as the seat portion 104. In some other examples, the back portion 106 may be rotatably connected to the seat portion 104, as shown in FIGS. 15C and 19. For example, the back portion 106 may be rotatably attached to a support bar or similar structure located near the back of or underneath the seat portion 104. In some embodiments, the seat portion 104 may be attached to the back portion 106 by one or more hinges, rotary joints, pivot joints, ball joints, or other rotatable connectors.

The back portion 106 may be rotated from a first position (e.g., the position corresponding to the seated position as shown in FIG. 1) to a second position corresponding to a recumbent position in which the support surface of the back portion 106 is generally parallel to and facing the same direction as the seating surface of seat portion 104. In this recumbent position, the seating system 100 may be used as, e.g., a gurney or a therapy table. Such a configuration may avoid the need to transfer the user out of the seating system 100 to another surface (e.g., a table or mat at a physical therapy office), thereby reducing the risk of injury which may occur during such transfer. In some examples, the back portion 106 may be rotated to one or more intermediate positions between the first position and the second position, allowing the seating system 100 to be configured in one or more reclined configurations, which may provide increased comfort as well as therapeutic benefits to the user. For example, an intermediate position may allow the user’s head to be slightly elevated, which may be useful for therapeutic purposes or user comfort. The back portion 106 may also be rotated to a position past perpendicular to allow the user’s head to be lowered, which may also be useful for therapeutic purposes of user comfort. A locking mechanism may hold the back portion 106 in the first position, the second position, an intermediate position, or a position beyond the second position when in a locked position (e.g., when the seating system 100 is moving) and may allow the back portion 106 to be moved between these positions when in an unlocked position (e.g., when the user would like to recline). FIGS. 16C and 16D shows the back portion 106 being rotated between a plurality of positions.

In some examples, the back portion 106 may additionally or alternatively be configured to rotate to a third position in which the support surface of the back portion 106 is generally parallel to and facing the opposite direction as the seating surface of the seat portion 104 faces. An example of the back portion 106 in the third position is shown in FIG. 15B. In this third position, the support surface of the back portion 106 is located directly over and facing the seating surface of the seat portion 104. The back portion 106 may thereafter be rotated with the seat portion 104 as described above for storage, as shown in FIG. 15C.

The back portion 106 may be constructed of any suitable material as discussed above in connection with the seat portion 104.

The primary wheels 108 may be positioned on either lateral side of the seating system 100 (and in particular on either lateral side of the seat portion 104) as shown in FIG. 1. The primary wheels 108 may be connected to the frame 102 via an axle 126 which connects the central hubs of the primary wheels 108 as shown in FIG. 1. In many cases, wheels on a conventional wheelchair may include a plurality of thin spokes that connect the central hub to the rim of the wheel. In embodiments of the disclosed subject matter, the primary wheels 106 may include a single bar 119 connecting the central hub to the rims of the wheels. The bar 119 may include a first spoke 120 connecting the central hub to a first location on the rim of the primary wheel 106 and a second spoke 122 connecting the central hub to a second location on the rim of the primary wheel 108, with the second location being located radially opposite (about 180 degrees from) the first location. In some other examples, the primary wheel 108 may include two bars (four spokes), three bars (six spokes), or four bars (eight spokes), and each bar may be evenly spaced from the others. In addition, the bar (as well as the spokes of which it is comprised) may have a comparatively thick width. The width may be, for example, at least one quarter inch (0.25″), at least one-half inch (0.5″), at least three quarters of an inch (0.75″), at least one inch (1″), at least one and a half inches (1.5″), at least two inches (2″), or at least three inches (3″). The use of fewer and wider spokes may decrease the risks of a user’s fingers or other objects getting caught in the spokes during operation. The bar and spokes may also be made of lighter materials, such as aluminum, titanium, or carbon fiber. Such a configuration may reduce the weight of the primary wheels 108 (and consequently the seating system 100), making transporting the seating system 100 easier, while still providing the needed stability, and may also reduce the dangers to the user associated with getting fingers caught in spokes.

The primary wheels 108 may also include a braking mechanism including a brake 124. The brake 124 may be located laterally outside of the rim of primary wheel 108, i.e., in the direction opposite the seat portion 104 as shown in FIG. 1. The brake 124 may have a circular shape and a diameter corresponding to the diameter of the rim of the primary wheel 108. In some embodiments, the brake 124 may be a disc brake.

The braking mechanism may also include a brake control 136 connected to the brake 124. The brake control 136 may control the brake 124 such that the brake control 136 activates the brake 124 when in first (inactive) position and the brake control 136 does not activate the brake 124 when in a second position. The brake control 136 may be located outside of the circumference of the primary wheel 108. In contrast, existing wheelchairs often have the brake control located inside the wheel which increases the danger that the user’s fingers may get injured when breaking.

In some embodiments, the brake control 136 may be located on the frame 102 and spaced apart from the primary wheel 108, as shown for example in FIG. 21. The brake control 136 may be located radially outside of the primary wheel 108 and may be spaced at least one inch from the primary wheel 108, at least two inches from the primary wheel 108, at least three inches from the primary wheel 108, at least four inches from the primary wheel 108, or at least five inches from the primary wheel 108. The distance between the brake control 136 and the primary wheel 108 may be adjustable to suit the needs of the user. A locking mechanism may be configured to hold the distance between the brake control 136 and the rim of the primary wheel 108 constant in a locked position (e.g., during use) and allow the distance between the brake control and the rim of the primary wheel 108 to be altered in an unlocked position (e.g., when the user or caregiver is configuring the seating system 100 upon initial receipt). The brake control 136 may allow the user to continuously maintain a grip thereon with decreased risk of injury. The outer surface of the brake control 136 may be curved to better fit the shape of a user’s hand during use and to increase comfort In some embodiments, the brake control 136 may be actuated to activate the brake 124 by pressing or pulling the outer surface of the brake control 136 towards the frame 102. In some embodiments, the brake control 136 may include a lever, a grab bar, a pressure sensor, or other mechanical or electrical elements. For example, the outer surface of the brake control 136 may be connected to a lever or may be configured to contact a pressure sensor when the outer surface is moved towards the frame 102.

In some embodiments, the brake control 136 may be a hydraulic brake control that uses hydraulic controls to activate the brake 124. In other embodiments, the brake control 136 may be a pneumatic brake control that uses pneumatic controls to active the brake 124. In other embodiments, the brake control 136 may use electric, mechanical, or electromechanical controls to activate the brake 124. In some embodiments, the brake control 136 may be connected to the brake 124 by one or more cables. The cables may be routed through the frame 102 and through one or more spokes of the primary wheel 108. In some embodiments, a seating system may include two brake controls 136, each of which is configured to activate a brake on a respective primary wheel. In some other embodiments, a seating system may include a single brake control 136 which may activate the brakes on both wheels. In seating systems with a brake on only one of the two primary wheels 108, the brake control 136 may activate the single brake.

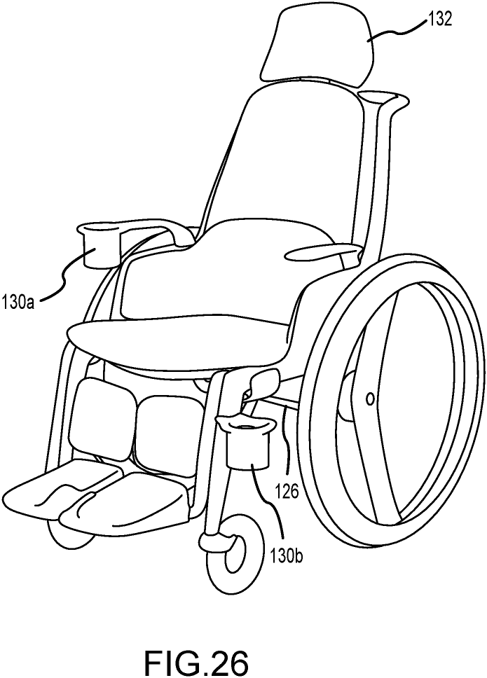

In some examples, the seating system 100 may include an axle 126 connected to the hub of each of the primary wheels 108 as shown, for example, in FIGS. 1 and 26. In some examples, the axle 126 may include two or more telescoping portions that allow the axle to be elongated or shortened for storage. For example, the axle 126 may be configured in a first position during operation through the use of a locking mechanism. When unlocked, the axle 126 may be elongated (to allow room for the other components to be rotated as necessary) and/or shortened (to reduce the profile of the seating system 100 for storage or transportation, as shown in FIG. 15D). In some examples, the axle 126 may include multiple operating positions to accommodate the user’s body, allowing the same seating system 100 to be used for a wide range of body types and other physical needs. The axle 126 may be formed in any desired shape or size. For example, the axle 126 may be linear or curved. In some examples, the axle 126 may include a first axle portion that connects the frame 102 to the first primary wheel 108 and a second axle portion that connects the frame 102 to the second primary wheel 108. One or both of the first axle portion and the second axle portion may be extendable such that the axle 126 can be shortened to decrease the distance between the first primary wheel and the second primary wheel. Other components (e.g., portions of the frame 102 extending from the first lateral side of the seating system 100 to the second lateral side of the seating system 100) may also be extendable to allow the distance between the primary wheels 108 to be decreased for storage and transportation, or to accommodate users with different body shapes.

The seating system 100 may further include secondary wheels 110. The secondary wheels 110 may be, for example, caster wheels, and may be provided primarily for stability.

The seating system 100 may also include armrests 112 located on either lateral side of the seat portion 104. The armrests 112 may include a support surface positioned generally parallel to the seating surface of seat portion 104 and facing in the same direction as the seating surface of seat portion 104 in the seating configuration as shown in FIG. 1. The armrests 112 may be rotatably connected to the frame 102. For example, the armrests 112 may be rotatably connected to the axle 126 on the interior side of the primary wheel 108 (i.e., the side of the primary wheel 108 opposite the brake). The armrests 112 may be configured to axially rotate about the axle towards the back of the seating system 100, e.g., in a direction away from the secondary wheels 110. In this manner, the armrests 112 may be rotated out of the way, e.g., when the user wishes to use a desk or to allow the user to be transferred into or out of the seating system 100 on a lateral side thereof. The armrests 112 may also be rotated in this manner for storage or transport purposes, as shown in FIG. 15D. In some examples, a locking mechanism may hold the armrests 112 in a first position in a locked position and allow the armrests 112 to rotate when in an unlocked position. In some other examples, the support surface of the armrests 112 may be configured to rotate about a supporting structure extending generally upward and parallel to the back portion 106, in which configuration the armrests 112 may not be connected to the axle 126 of the seating system 102.

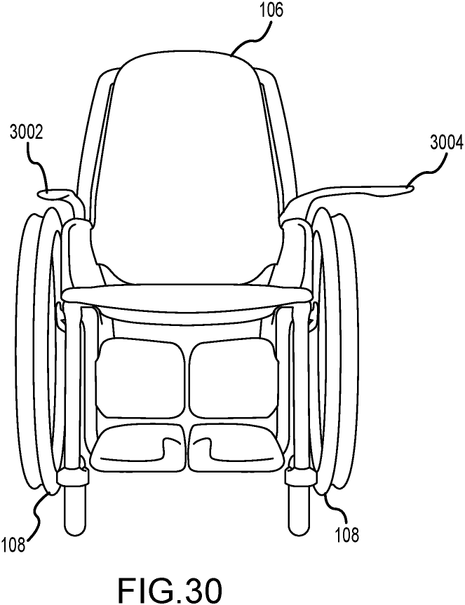

FIG. 30 shows an example of a seating system 3000 in which the armrests 112 are configured to rotate about a supporting structure between a first position 3002 and a second position 3004. In the first position 3002, the armrest 112 extends forward in a direction generally perpendicular to the back portion 106 and generally parallel with the plane of the primary wheels 108. In the second position 2004, the armrest 112 extends laterally in a direction generally parallel with the back portion 106 and generally perpendicular to the plane of the primary wheels 108. This configuration also allows the armrests 112 to be rotated out of the way.

In some examples, the armrest 112 may include indicator lights that may be viewable to the user of the seating system 100. For example, the armrests 112 may include a power light with two or more states. For example, the power light could have a first state (e.g., no light) indicating that the seating system is not powered on and a second state (e.g., white light) indicating that the seating system is powered on. In some other examples, the power light could have a first state (e.g., no light) indicating that the seating system is not powered on, a second state (e.g., green light) indicating that the seating system 100 is powered on and charged above a threshold value (e.g., 20% battery), and a third state (e.g., red light) indicating that the seating system 100 is powered on but is not charged above a threshold value (e.g., 20%).

In some examples, the seating system 100 may also include a plurality of integrated biometric sensors. The armrest 112 may include a light corresponding to the status of the sensor. For example, a heart rate indicator light may correspond to the status of a heart rate sensor. A green light may indicate a safe condition, indicating that the user’s heart rate is within a safe range (or ranges), while a red light may indicate an emergency condition, indicating that the user’s heart rate is with a range (or ranges) that are considered dangerous. The ranges corresponding to a safe condition and an emergency condition (and any other desired condition, such as a warning condition that may correspond to a yellow light) may be selected by the user’s medical or other care provider and stored in a memory of the seating system 100. In some examples, a set of default ranges may be provided. The default ranges may be stored in a memory of the seating system. In some examples, the default ranges could be modified by the medical or other care provider. In some examples, the default ranges may be determined based on the user’s medical condition or other variables. For examples, a sensor may have a first set of default ranges associated with a first one or more medical conditions, a second set of default ranges associated with a second one or more medical conditions, and a third set of default ranges for situations not associated with either the first or second set of one or more medical conditions. Indicator lights may be provided on the armrests 112 for one or more integrated sensors, including the sensors described in greater detail herein. One or more processors associated with the seating system 100 may be configured to receive a sensor measurement, compare the sensor measurement against a set of ranges stored in memory, and control the indicator lights based on whether the sensor measurement is within the set of ranges.

In some examples, the armrest 112 may alternatively include a screen that provides information such as power information or sensor information to the user. In some examples, a screen may additionally or alternatively be located in other locations of the seating system 100, such a behind the back portion 104 such that the screen may be viewed by a caregiver. In some examples, the screen may be configured as a tablet in electronic communication (which may be wired or wireless) with the seating system 100. The screen may also be associated with a removable holder that may be attached when the user desired to view the screen and detached when the user does not need the screen.

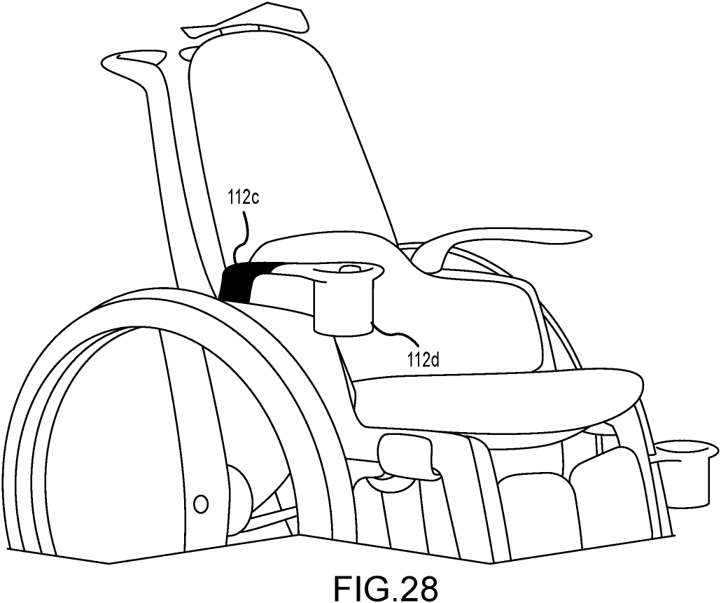

In some examples, one or more armrests 112 may include an attachment portion 112c and a removable portion 112d, as shown in FIG. 28. The attachment portion 112c may remain permanently connected to the seating system 100 and may include an interface that allows the removable portion 112d to be selectively removed from and attached to the seating system 100. Alternatively, the entire armrest 112 may be removable and the interface for the removable armrest may be on the frame 102 or other component of the seating system 100. In this manner, the user or caregiver may modify the seating system 100 to suit the needs of the user. The removable portion 112d shown in FIG. 28 includes a cupholder. The removable portion 112d may be removed and replaced with other removable portions such as, by way of example, a support armrest (e.g., the armrest 112 shown in FIG. 1), a tray (which may, for example, provide a surface for the user to place food or use for writing), a stand (which may, for example, provide a place for the user to prop up and read books, magazines, or a tablet), or any other useful attachments. In some examples, the interface of the attachment portion 112c may include electronic connections such as power and wired communications ports such that the attachment portion 112c can provide electronic communication with appropriate removable portions 112d. In other examples, the removable portion 112d may be powered by an internal power source such as a rechargeable battery and/or may communicate with the seating system 100 through wireless communication interfaces. In this manner, the removable portion may include an integrated touch screen or other electrical components such as the electrical components described herein. In other examples, the attachment portion 112c may be located on a side opposite an armrest 112 that includes indicator lights, touchscreen or other displays, control buttons, a joystick, or the like, as described herein, such that no electrical communication or power is needed for the removable portion 112d.

The seating system 100 may also include footpads 114 which may be connected to the frame 102 via a support rod 128 that extends generally downward from the frame 102 in a seating position as shown in FIG. 1. In some examples, the support rod 128 may have an adjustable length. For example, the support rod 128 may include two or more telescoping rods. A locking mechanism may hold the telescoping rods in a defined relation in a locked position and allow the support rod 128 to be extended or shortened in an unlocked position. In this manner, the height of the footpads 114 may be adjusted to accommodate users of various heights and/or leg lengths.

The footpads 114 may be rotatably connected to the support rod 128. For example, the footpads 114 may be rotated from a first position that is generally perpendicular to the support rod 128 (i.e., that is generally perpendicular to direction in which the support rod 128 extends) and the calf pads 116, as shown in FIGS. 15A and 15B, to a second position that is generally parallel to the support rod 128 and the calf pads 116, as shown for example in FIG. 15C. In this second portion, the support surface of a footpad 114 may face towards a support surface of a calf pad 116. In contrast to designs where a footpad 114 rotates axially around a support rod 128, this configuration decreases the risk that the footpad 114 will rotate back under foot while the user is attempting to exit the seating system 100. In some examples, the footpads 114 may also be rotated to other positions (e.g., a position which causes the user’s foot to be positioned either at a slight incline or a slight depression) for comfort and/or therapeutic purposes.

The support rods 128 may be rotatably or pivotably connected to the frame 102. In some examples, the support rods 128 may be rotated from a first position in which they extend generally downward from the frame 102 to a second position that is generally parallel to and underneath the seat portion 104. The support rods 128 can thereafter be rotated to a stored position when the seat portion 104 is rotated as described above. In some other examples, the support rods 128 may remain extending downward from the frame in a stored position as shown in FIG. 15D. As further shown in FIG. 15D, the footpads 114 may further rotate to a third position that is generally perpendicular to both the first position and the second position for storage or transport.

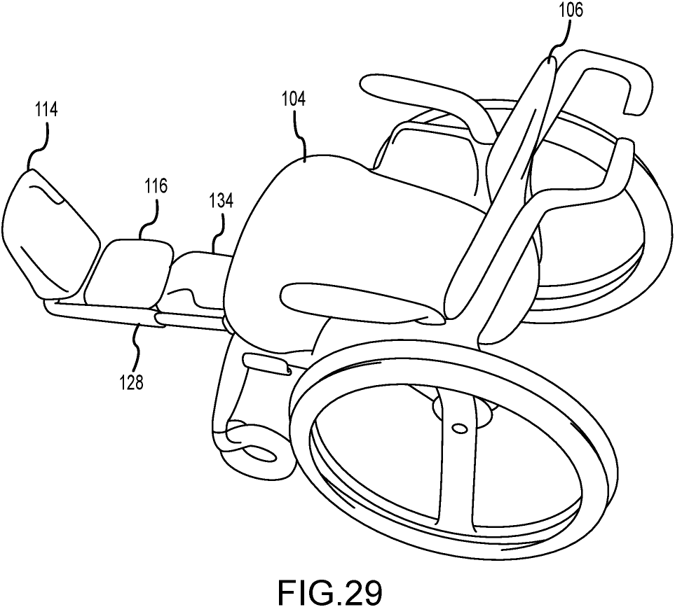

In some other examples, the support rods 128 may be rotated from the first position to a third position that is generally perpendicular to the seat portion 104 and extends outward therefrom, as shown in FIG. 29. In this third position, the support rods 128 may extend in a direction parallel to the seat portion 104 and the back portion 106 when the seating system 100 is configured in the recumbent configuration. The footpads 114 may also be configured to rotate to a position parallel to the support rods 128, the seat portion 104, and the back portion 106 when the seating system 100 is configured in the recumbent configuration, providing support for the user along a full extension in the recumbent configuration (which may be denoted the fully recumbent configuration). The footpads 114 may alternatively be left in a position generally perpendicular to the support rods 128 when the seating system 100 is configured in the recumbent configuration. In the recumbent position, the support rods 128 may be extended or shortened to move the footpads 114 for therapeutic purposes. As further shown in FIG. 29, the seating system 100 may also include one or more additional pads 134, which may be detachable and may be attached when the support rods 128 are extended or when further support is needed. For example, further support may be needed when the seating system 100 is in the recumbent configuration to support a user’s thigh.

In some examples, the support rods 128 may also be rotated to other positions for user comfort or therapeutic purposes.

In some examples, the support rods 128 may also be connected to a control mechanism which will assist the user in lifting their legs, e.g., from the seated position to a fully recumbent position. In some examples, the control mechanism may be a lever that can mechanically assist the user in lifting their legs, which may be operated by the caregiver. In some other examples, the control mechanism may be a lever or electronic control mechanism (e.g., a button or switch on the armrest 112 of the seating system 100 or a remote application or other computer program that can send a signal to the seating system 100) which electronically assists the user in lifting their legs.

The seating system 100 may also include calf pads 116 which may be connected to the support rod 128. The calf pads 116 may be connected to an adjustable portion of the support rod 128. In some examples, the height of the calf pads 116 may be adjusted independently of the height of the footpads 114. For example, the calf pads may be slidably connected to the support rod 128. The calf pads 116 may be provided for comfort and may also provide support, e.g., when the seating system is configured in the fully recumbent configuration. The calf pads 116 may be rotatably attached to the support rods 128 such that they can be rotated out of the way so that the user need not lift their legs over the calf pads 116 when getting into and out of the seating system 100. For example, the calf pads 116 may be rotatably connected to the support rods 128 such that they can be rotated from a first position (as shown in FIG. 1) to a second position generally perpendicular to the first position with the top of the calf pad 114 facing the opposite lateral side of the seating system 100. In this second position, the calf pad 114 may be located underneath the seat portion 104 and between the support rod 128 and the axle 126 (e.g., when the seating system 100 is in the seated configuration). In some other examples, the calf pad 114 may face laterally outward (i.e., toward the primary wheel 108 on the same side of the seating system 100) when in the second position, such that the calf pad 114 is located in front of the support rod 128, and on the opposite side of the support rod 128 form the axle 126. After the user has situated himself or herself in the seating system 100, the calf pad 114 may be rotated 90 degrees back to its original position to provide support behind the user’s calf. The second position may also be used for storage and transportation.

The seating system 100 may also include a push bar 118, which may be used by the caregiver to steer the seating system 100. The push bar 118 may also be used to lock one or more of the locking mechanisms described herein, ensuring that the adjustable components are locked in place when the seating system 100 is in motion. In some examples, the push bar 118 may be rotatably connected to the frame 102 so that the push bar 118 can be rotated, e.g., for putting the seating system 100 into a recumbent position, for storage or to provide varying use positions depending upon the height of a care giver, as shown for example in FIGS. 16B and 16D. The handles of the push bar may also be rotatable relative to the remainder of the push bar, which may be useful in storage and transportation.

The seating system 100 may also include additional features that provide operability and safety benefits. For example, the seating system 100 may also include one or more lights. In some examples, one or more lights may be positioned on the footpads 114 to provide illumination of the ground in front of and underneath the seating system 100. In some examples, one or more lights may be positioned on the secondary wheels 110, such as on the caster portion of the caster wheels. In some examples, the one or more lights may be positioned on the central hub of the primary wheels 108 and/or around the rim of the primary wheels 108 (e.g., on the rim or brake), which will provide illumination to the user in operating the seating system 100. In some examples, one or more lights may be positioned on the push bar 118. In some examples, one or more lights may be positioned on the back of the seating system 100 (such as on the rear-facing portion of the back portion 106 and/or the rear-facing portion of the frame 102), which both provides illumination behind the seating system 100 and also may serve to provide safety from, e.g., bicycles or cars that may be approaching the seating system 100 from behind. For example, these lights may serve a similar function as the taillights on a car. The lights may be turned on by the user or caregiver (e.g., using a button on the armrests) or may be activated automatically (e.g., based on light sensors, motions sensors (which may sense either external motion or when the seating system 100 is moving), and/or time). In addition to or as an alternative to any rear-facing lights, the seating system 100 may include one or more reflectors, including on the rear-facing portions of the seating system 100 and/or on the lateral sides of the seating system 100, which reflectors will serve a similar function as the reflectors on a bicycle.

In some examples, seating system 100 may be a manual wheelchair. In other examples, the seating system 100 may be a power wheelchair that is propelled by electrical power. In some embodiments, the seating system 100 may include a motor or other propulsion system, which may be stored underneath the seat portion of the wheelchair.

In some examples, the seating system 100 may include one or more cupholders 130, as shown in FIG. 26. For example, a cupholder 130a may be formed as part of one of the armrests 112. In other examples, a cupholder 130b may also or alternatively be attached to or formed as part of the support rods 128 or an adjacent part of the seating system 100 (e.g., a part of the frame of the seating system 100). The seating system 100 may also include a cupholder or other storage area behind the back portion 106 for use by a caregiver.

The seating system 100 may also include a headrest 132 located above the back portion 106, as shown in FIG. 26. The headrest 132 may be rotated along with the back portion 106, e.g., to put the seating system 100 in a stored or recumbent configuration as described herein. In some examples, the headrest 132 may be slidably connected to the back portion 106 to allow the headrest 132 to be adjusted based on the height of the user. Such slidable configuration may also allow the headrest 132 to be moved either partially or entirely behind or in front of the back portion 106 for storage. In some other examples, the headrest 132 may be rotatably connected to the back portion 106, allowing the headrest 132 to be rotated such that the support surface of the headrest 132 faces towards the support surface of the back portion 106 for storage. The headrest 132 may also be rotated in the opposite direction for storage such that the back surface of the headrest 132 faces towards the back surface of the back portion 106.

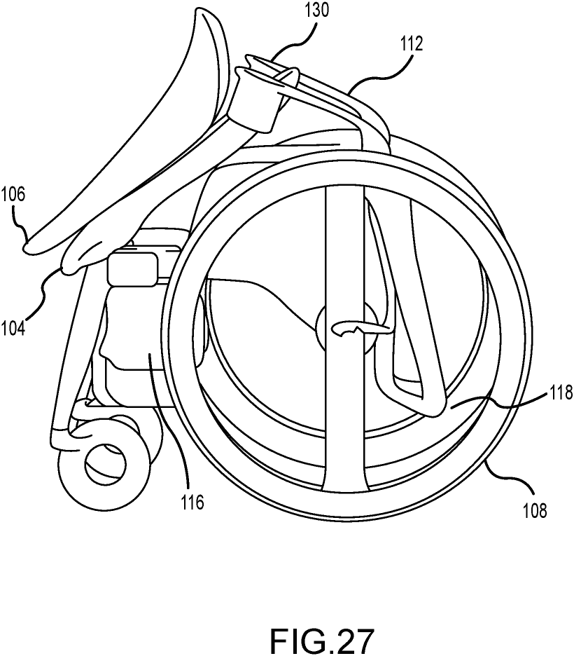

FIG. 27 shows another embodiment of a seating system in accordance with the disclosed subject matter. The seating system shown in FIG. 27 provides an alternative method for collapsing a seating system in accordance with the disclosed subject matter. In this embodiment, the back portion 106 may be rotated from a seating position generally perpendicular to the seat portion 104 to a storage position generally parallel with the seat portion 104. The back portion 106 may be rotatably connected to the seat portion 104 as shown in FIGS. 15C and 19, although that connection is not shown in FIG. 27. The seat portion 104 may be detachably attached to the frame of the seating system at a side of the seat portion 104 proximate the back portion 106 (i.e., at the back of the seat portion 104). For example, the seat portion 104 may be detachably attached to the frame of the seating system at locations proximate the back portion 106 on both the first frame portion and the second frame portion of support frame 102 as shown in FIG. 1. The seat portion 104 may also be rotatably attached to the frame of the seating system at a side of the seat portion 104 remote from the back portion 106 (i.e., at the front of the seat portion 104). For example, the seat portion 104 may be rotatably connected to the frame of the seating system at locations remote from the back portion 106 on both the first frame portion and the second frame portion of support frame 102 as shown in FIG. 1. In some embodiments, the seat portion 104 may be attached to the frame 102 by one or more hinges, rotary joints, pivot joints, ball joints, or other rotatable connectors. When the seat portion 104 is detached from the frame at its back edge, both the seat portion 104 and the back portion 106 may be tilted forward (i.e., angle such that the back of the seat portion 104 is elevated relative to the front of the seat portion 104) to allow the primary wheels 108 to be pushed together (e.g., by shortening an extendable axle 126). As shown in FIG. 27, the back edge of the seat portion 104 (i.e., the edge of the seat portion 104 proximate the back portion 106 when the seat portion 104 is in the seated configuration, as shown in FIG. 1) may be elevated relative to the front edge (i.e., the edge of the seat portion 104 opposite the back edge) when the seating system is collapsed. In this alternative storage configuration, the seat portion 104 and back portion 106 may be located outside of the primary wheels 108 rather than between them as shown in FIG. 15D. As with the stored configuration described in FIG. 15D, the push bar 118 may be rotated and stored behind the frame of the seating system. The footpads 114 may also be rotated from a first position generally perpendicular with the calf pads 116 and generally perpendicular with the support rods 128 to a second position generally parallel with the calf pads 116 and the support rods 128, e.g., where the support surface of the footpads 114 face the support surface of the calf pads 116. The footpads 114 and calf pads 116 may thereafter be rotated from a first position to a second position generally perpendicular to the first position. The second position may be, for example, between the support rods 128 and the axle 126 of the seating system, as shown in FIG. 27, or may be in front of the support rods 128.

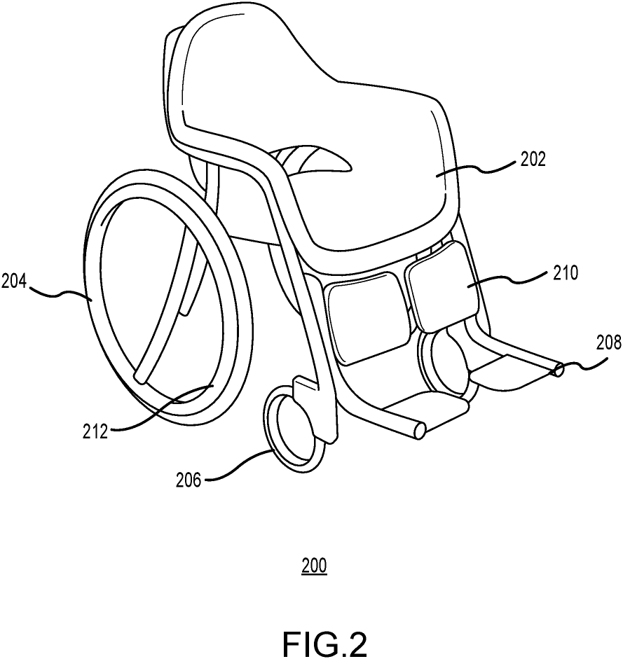

FIG. 2 shows another embodiment of a seating system in accordance with the disclosed subject matter. The seating system 200 may include a seating portion 202, primary wheels 204, secondary wheels 206, footpads 208, and calf pads 210. The seating system 200 may use light materials and a minimalist design, providing a seating system that is both comfortable and significantly lighter than existing systems. The lighter weight may be particularly important where a user may need to move their own seating system (e.g., because no caregiver is available) or where the caregiver also has physical limitations that do not allow them to lift heavy objects (e.g., where the caregiver is the user’s elderly spouse).

The seating portion 202 may include a single-piece seating portion. The seating portion 202 may be ergonomically designed and may be constructed of suitable materials such as, for example, plastic woven mesh or lightweight metal such as titanium or aluminum. In other embodiments, the seating portion 202 may be a lightweight seating portion (which may be constructed of, e.g., plastic woven mesh) including two or more pieces.

The primary wheels 204 may be similar to the primary wheels 108 as described above in connection with FIG. 1. In some examples, the seating system 200 may not include an axle connecting the wheels. Instead, the central hub of the primary wheel 204 may be directly connected to the lightweight frame of seating system 200.

The secondary wheels 206 of seating system 200 may be similar to the secondary wheels 110 as described above in connection with FIG. 1.

The footpads 208 may be substantially similar to the footpads 114 as described above in connection with FIG. 1. In some examples, the footpads are not adjustable and cannot be moved, which eliminates the additional parts (and weight) needed to enable such functionality. In some other examples, the footpads 208 may be connected to the lightweight frame via lightweight, telescoping support rods to allow for adjustment of the height of the footpads 208.

The calf pads 210 may be substantially similar to the calf pads 116 as described above in connection with FIG. 1. In some examples, the calf pads 210 may be constructed with the same materials as the seating portion 202. The seating system 200 may also include additional components as described above in connection with FIG. 1, including for example a brake 212 for a first primary wheel located on the side of the first primary wheel opposite the second primary wheel.

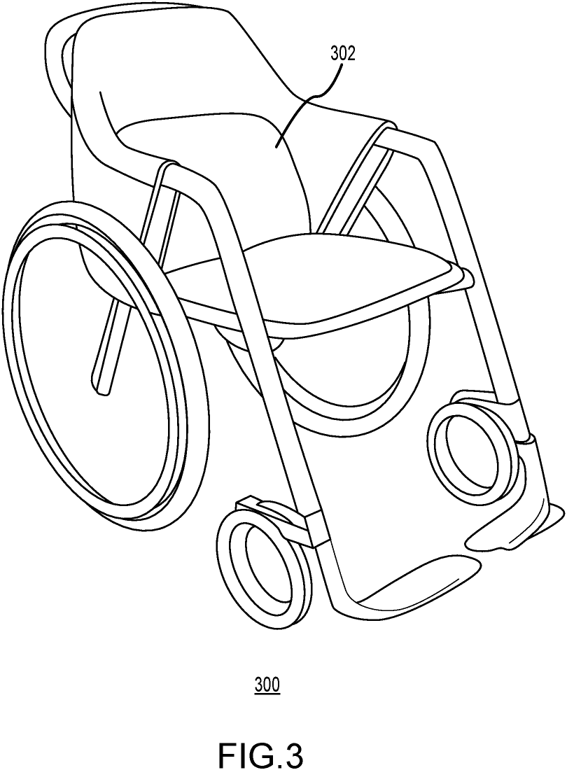

FIG. 3 shows another embodiment of a seating system in accordance with the disclosed subject matter. The seating system 300 is similar to the seating system 200 as discussed above in connection with FIG. 2. Although not illustrated in FIG. 3, the primary wheels may connect to the seating portion of the wheelchair via a single bar including a first spoke and a second spoke, as shown in FIG. 2. The seating system 300 may also include an integrated cushion 302 which may include one or more sensors including, for example, a pressure sensor, a moisture sensor, a heart rate sensor, and/or a temperature sensor. The cushion 302 may include one or more inflatable chambers that may be separately controlled as described herein.

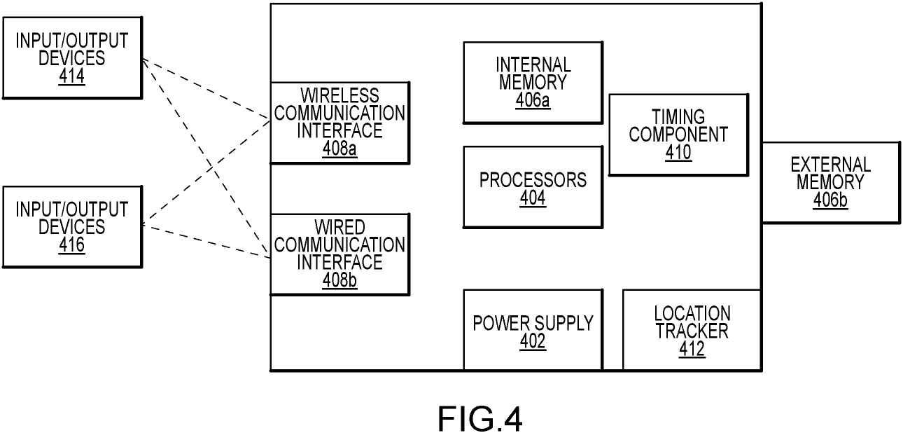

FIG. 4 is a block diagram of a circuit board 400 for a seating system in accordance with embodiments of the disclosed subject matter. The circuit board 400 may include a power supply 402, one or more processors 404, memory 406, communication interfaces 408, a timing component 410, and a location tracker 412. The circuit board may be, for example, a printed circuit board (PCB). Although FIG. 4 shows components located on the circuit board 400, one or more of these components may be moved off of the circuit board 400 without departing from the scope of the disclosed subject matter.

The circuit board 400 may include a power supply 402 that provides power to seating system. The power supply 402 may include, for example, a battery such as a rechargeable battery. In some embodiments, the rechargeable battery could be connected to the circuit board 400 but be located in another location such as underneath the seat portion 104 of wheelchair 100 as shown in FIG. 1. In some examples, the power supply 402 may include an interface where an external power supply may be connected to recharge the rechargeable battery. For example, the interface may provide a connection for an electrical cord that can be plugged into a wall outlet to allow the rechargeable battery to be recharged using the power grid. In some examples, the power supply 402 can include or be connected to a generator that can provide a power source for the seating system in an emergency. In some examples, the power supply 402 can include or be connected to photovoltaic cells that may be positioned on the seating system. The photovoltaic cells may be positioned on areas of the seating system that may be exposed to sunlight such as, for example, the back of the seating system (e.g., on the rear side of back portion 106) or on the top of the armrests (e.g., on the top of armrests 112a and/or 112b). The photovoltaic cells may also be placed in other locations.

The circuit board 400 may also include one or more processors 404. The processors 404 may be configured to execute instructions stored in one or more memories 406 of the seating system. For example, the processors 404 may be configured to execute instructions to carry out any of the operations of the seating system as described herein, including but not limited to adjusting the pressure of a cushion based on pressure sensor measurements, detecting an alert condition based on sensor measurements, communicating alert conditions to remote devices, and transmitting sensor data to a backend system for storage.

The circuit board 400 can include one or more memories 406. The one or more memories 406 can include volatile memory and/or non-volatile memory. In some examples, the one or more memories 406 may include internal memory 406a (which may be referred to as internal memory units or IMU) such as, for example, RAM, ROM, or flash memory. In some examples, the one or more memories 406 may also include external memory 406b, such as a data storage device (e.g., an SD card) that may be removably connected to a memory interface of the seating system (e.g., an SD card slot). In some other examples, the external memory 406b may include embedded storage devices such as hard disks or solid state drives. The one or more memories 406 may be communicably connected to the processors 404 such that the processors 404 can read from and write to the memories 406 as needed.

The circuit board 400 may further include one or more communication interfaces 408 that allow communication between the processors 404 and external systems.

In some embodiments, the circuit board 400 may include one or more wireless communication interfaces 408a that allow the seating system to communicate with other components of the seating system and/or external systems over a wireless connection. The wireless communication interfaces 408a may include one or more of a Bluetooth communication unit including a Bluetooth antenna, a WiFi communication unit including a WiFi antenna, a cellular communication unit including a cellular antenna, an infrared communication unit, or any other type of wireless communication unit known in the art.

In some embodiments, the circuit board 400 may include one or more wired communication interfaces 408b that allow the processors 404 to communicate with other components of the seating system or to communicate with external systems including input devices. For example, the wired communication interfaces 408b may include one or more Universal Serial Bus (USB) ports. Other wired communication protocols may also be used, including for example Display Serial Interface (DSI), Inter-Integrated Circuit (I2C), or any other type of wired communication protocol as known in the art.

The circuit board 400 may include a timing component 410 such as a real time clock (RTC). The timing component 410 may be used for various purposes, including for example to measure time for use in determining a user’s heart rate in beats per minute, measuring the time duration a user is positioned in or on the seating system, or measuring the time duration the seating system is positioned in a specific orientation. In some examples, the processors may be configured to provide nudges to a user based at least in part on the timing component 410. For example, if the processors determine that the user has been positioned on the seating system for a time duration that meets or exceeds a threshold time duration, the processors may suggest that the user stand up. In some other examples, if the processors determine that the user has been positioned in a particular seating position for a time duration that meets or exceeds a threshold time duration, the processors may suggest the user change to a different seating position. In some examples, the suggestion may be a textual message, e.g., transmitted through a display screen viewable to the user or caregiver, or an audio message provided to the user or caregiver through a speaker.

The circuit board 400 may include a location tracker 412. The location tracker 412 may be a global positioning system (GPS) unit. The GPS unit may include a GPS antenna. In some examples, the location tracker 412 may use another global navigation satellite system (GNSS) such as, for example, the Galileo system. The location tracker 412 may be used to locate the seating system if the seating system is lost, if the caregiver loses track of the user, or if the caregiver, medical provider, or a family member or friend of the user wants to check in on the location of the user. For example, the location tracker 412 may allow a caregiver to track the location of the seating system through a mobile application on the caregiver’s device. The location tracker 412 may be used in combination with a weight sensor and/or pressure sensor to confirm that the user is still in the seating system. Alternatively, if the user is no longer in the seating system, historical data from the location tracker 412 may be combined with historical data from the weight sensor and/or pressure sensor to determine where the seating system was located when the user exited the seating system.

By using the communication interfaces 408, the processors 404 may be able to communicate with other components of the seating system and/or external systems. For example, the communication interfaces 408 may provide communications between the processors 404 and one or more input/output devices 414 and/or one or more sensors 416.

The input/output devices 414 may allow the user, caregiver, medical provider, or another interested party to provide information to or receive information from the seating system. For example, the input/output devices 414 may include one or more input devices such as a keyboard, a touchscreen, a joystick, microphone, or the like. For example, the user or caregiver may use a keyboard or a touchscreen to assist in the setup process, to control the operation of the seating system, to request information about the status of the user or the operation of the seating system, and/or to input information, including settings, into the seating system. In some examples, the seating system may include a joystick or other assistive technology device that allows the user to steer the seating system. In some examples, the seating system may include a microphone that allows for voice commands using voice recognition technology as known in the art, which may be beneficial if the user has a medical condition that prevents them from manually inputting information. In some examples, the seating system may include an input panel or touch screen, e.g., on one or both armrests 112 of wheelchair 100 as described in connection with FIG. 1.

In some embodiments, the input/output devices 414 may include one or more output devices such as a display screen or a speaker. For example, the user and/or caregiver may use a display screen (e.g., an LCD display) to review requested information about the status of the user (e.g., to monitor the user’s health) and/or the operation of the seating system. In some examples, the seating system may include one or more speakers to provide audio information to the user and/or caregiver, which may be beneficial if the user has a medical condition that prevents them from viewing visual (e.g., textual) information on a display screen. In some examples, the seating system may include one or more lights or other visual indicators that may provide information to the user and/or caregiver (e.g., lights that indicate the power status of the seating system and/or whether an alert condition exists). Examples of indicator lights are described in connection with FIG. 1.

In some embodiments, the input/output devices 414 may include one or more remote computing devices. For example, the input/output devices 414 may include a remote computing device controlled by a medical provider through which the medical provider can access medical records stored in the one or more memories 406 and/or through which the medical provider can store information to the one or more memories 406. For example, the medical provider may be able to set or update threshold values for emergency conditions or alerts based on the user’s medical condition. In some examples, the input/output devices 414 may include a software application running on a mobile device that allows the caregiver, medical provider, or other interested party (e.g., family member or friend) to monitor the user’s condition.

In some embodiments, the processors 404 may be configured to send alerts to one or more specified external systems when a predetermined condition occurs. The condition may be an emergency or non-emergency condition. An emergency or non-emergency condition may be an emergency or non-emergency event. An emergency or non-emergency condition may occur based upon sensed biometric information or sensed seating system information. An example of an emergency condition includes but is not limited to a condition of the user (e.g., heart rate) or a condition of the seating system (e.g., an overturned seating system). An example of a non-emergency condition includes but is not limited to a condition of the seating system (e.g., low power storage). The processors 404 may determine that an emergency or non-emergency condition exists based on data received from sensors 416. For example, the processors 404 could be configured to send an alert to one or more of the following external systems when a predetermined condition occurs: one or more computing systems associated with the caregiver (e.g., a display screen connected to the seating system or a mobile communication device associated with the caregiver), one or more computing systems associated with the medical or other care provider (e.g., a computer at the medical provider’s office or a mobile communication device associated with the medical provider), and/or one or more computing systems associated with family members or other individuals specified by the user (e.g., a mobile device associated with the user’s child or spouse, or a close friend).

The sensors 416 may include one or more biometric sensors. For example, the sensors 416 may include a sensor that measures a user’s blood oxygen level such as a pulse oximeter. In some examples, the pulse oximeter may be located on the seating system. For example, the pulse oximeter may be integrally formed on an armrest of the seating system. In other examples, the pulse oximeter may be removably connected to the seating system via a communication interface. In some other examples, the pulse oximeter may communicate with the seating system via wireless communications.

In some embodiments, the sensors 416 may include a heart rate sensor. The heart rate sensor may be, for example, an ECG or EKG sensor. In some other examples, heart rate sensor may provide raw information (e.g., voltage readings) and the heart rate may be determine based on the timing component 410. The heart rate sensor may include one or more electrical leads integrated into the seating system, e.g., in armrest, the seat portion, the back portion, and calf pads of the seating system.

In some embodiments, the sensors 416 may include a temperature sensor. The temperature sensor may include thin film sensors integrated into the seating system, e.g., in the seat portion, back portion, or armrest of the seating system. The temperate sensors may be configured to monitor body temperature averages over a period of time.

Other types of biometric sensors may also be used in accordance with the disclosed subject matter. For example, the sensors 416 may include a blood pressure sensor, a blood glucose sensor, or any other biometric sensor as known in the art. The biometric sensors may be integrated with the seating system, removably attached to the seating system, or wirelessly connected to the seating system.

The sensors 416 may also include a sensor for measuring the weight of the user. This sensor may be located on or under the seat of the seating system. The sensors 416 may also include one or more pressure sensors, which may be located on the seat portion of the seating system. The pressure sensors may be, for example, load cells or other transducers. The sensors 416 may also include sensors for measuring the speed and/or orientation of the seating system. For example, the sensors 416 may include a gyroscope for measuring the orientation of seating system. The sensors 416 may also include one or more motion sensors. The sensors 416 may include a sensor that measures the ambient temperature where the seating system is located.

In some embodiments, the sensors 416 may include a moisture sensor. In some examples, a cushion of the seating system may include thin film sensor elements to allow the seating system to detect moisture on the surface of the cushion, which may indicate a medical or comfort problem for the user. In some examples, the seating system may identify one or more areas of potential danger for skin breakdown, e.g., bed sores, skin tears, or infections based on the moisture sensor and/or pressure sensors. For example, the processors 404 may identify a location of potential danger when a moisture measurement at the location meets or exceeds a moisture threshold and a pressure measurement at the location meets or exceeds a pressure threshold. The seating system may communicate information identifying the location to the caregiver or medical or other care provider to assist them in caring for and providing medical treatment to the user.

One or more of the processors 404 may be configured to receive data from the one or more sensors 416 and control the operation of the other electronic elements of the seating system based on the received data. The processors 404 can receive data gathered by the one or more sensors 416 and store the data in memory 406. The processors also can retrieve data gathered by the one or more sensors 416 from one or more of the memory units 406. The one or more memory units 406 may be configured to store one or more default or configurable settings of the seating system such as the ranges associated with an indicator light and/or sensor 416 as discussed above in connection with FIG. 1. The one or more memory units may also be configured to store historical sensor readings. In some examples, the one or more memory units may be configured to store historical sensor readings for a set period of time, e.g., two weeks. In some other examples, the one or more memory units may store historical sensor readings only until that information is transferred to a backend system. Such information may be transferred to the backend system for storage in real-time, at pre-determined intervals (e.g., every minute or every hour), or on-demand.

One or more features of the seating system may be controlled based at least in part on the sensor data. For example, the one or more memories 406 may include instructions that will cause the processor to perform certain actions if a condition occurs. For example, the seating system may automatically stop the movement of the seating system (e.g., by shutting down the motor or other propulsion system, or applying an electronic brake) if the detected weight suddenly drops to zero, indicating that the user is no longer in the seating system, or may be configured to sound an alarm or issue an alert to the caregiver, medical provider, or another person if a gyroscope indicates that the seating system has overturned.

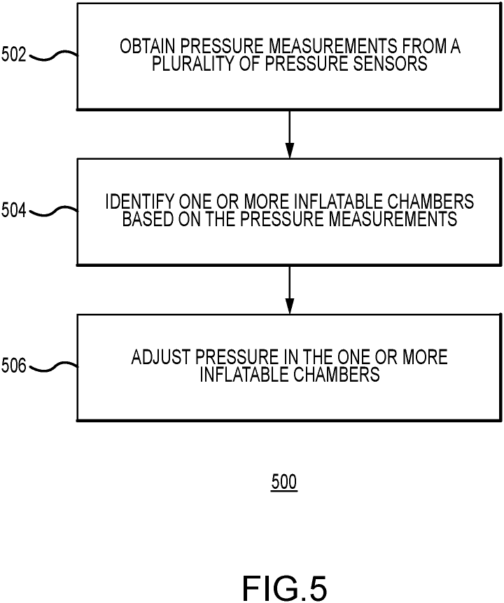

FIG. 5 is a flowchart 500 describing a method for adjusting the pressure of a cushion on a seating system in accordance with embodiments of the disclosed subject matter. The cushion may include one or more inflatable chambers. For example, the cushion may include multiple bladders. The bladders may be, for example, insert molded, co-molded, or otherwise formed from synthetic materials which may be sealed using solvents, heat, or other known sealing methods. In some examples, the cushion may include separate chambers (e.g., bladders) formed in different patterns. The patterns may vary based on the user’s anatomy, the user’s age, the user’s gender, or the user’s medical condition. In some examples, the bladder pattern may be subdivided into a lateral left portion and a lateral right portion, each with multiple bladders, or it may comprise a concentric circle pattern, wherein each circle or annular ring has one or more bladder sections. In some examples, each inflatable chambers may be approximately the same size and shape. In other examples, each inflatable chamber may have a different size or shape. However, the disclosed subject matter may be used with any configuration of inflatable chambers or bladders.

In embodiments of the disclosed subject matter, the seating system may include a fluid-filled (e.g., gas-filled or liquid-filled) cushion. The cushion may be located in the seat area of the seating system and/or on the back of the mobile seating system (i.e., the area of the seating system against which the user rests his or her back during operation of the seating system). For example, the cushion may be located on seat portion 104 and/or back portion 106 of seating system 100 as shown in FIG. 1. Fluids-filled cushions may alternatively or additionally be located on any area of the seating system against which a portion of the user’s body may rest, either when the user is seated, lying prone, or otherwise using the seating system as described herein. In some examples, the seating system may be a gurney and a majority or even the entirety of the gurney may be covered by a fluid-filled cushion with one or more inflatable chambers as described herein. The cushion may be integrally formed with a surface (such as, for example, seat portion 104 as shown in FIG. 1). In other examples, the seating system may be configured to accept one or more cushions and each cushion may be provided with one or more interfaces for an electrical and/or data connection and an air or other gas connection. In some other examples, the cushion may be configured to operate independently (e.g., the cushion may have its own separate power source) but may be paired with the seating system via a wireless connection.

In some embodiments, the seating system may include a removable portion that may be transferred between different locations, e.g., between a first wheelchair and a second wheelchair or between a wheelchair and another seating apparatus (e.g., a seat on an airplane so that the removable portion may be used during travel). The removable portion of the seating system may include one or more cushions as described herein. In some examples, the removable portion may include a memory that stores user settings and/or software instructions for the removable portion (e.g., user pressure preferences for a fluid-filled cushion). For example, the memory may include instructions for adjusting the pressure of the cushion based on pressure readings in accordance with FIG. 5. In some examples, the removable portion may include a wireless communication module or unit (e.g., a Bluetooth unit including a Bluetooth antenna or a WiFi unit including a WiFi antenna) such that the user can control the removable portion remotely (e.g., to adjust the pressure of the cushion and/or request massage functionality as disclosed herein).

The method described in flowchart 500 begins with obtaining pressure measurements from a plurality of pressure sensors located on and/or integrated with the cushion at 502. In some examples, the pressure sensors may be surface pressure sensors that measure the pressure at a surface on the surface of the cushion. For example, the surface pressure sensor may be an electrical pressure sensor that covers the seating side of the cushion. In some other examples, the pressure sensors may be chamber pressure sensors that measure the pressure of the air or other gas in the inflatable chambers of the cushion. The chamber pressure sensors may be situated inside each chamber and measure the pressure of the fluid located therein. In some other examples, the pressure sensors may be chamber pressure sensors that measure the pressure of the air or other fluid in the inflatable chambers. In some embodiments, the cushion may include both surface pressure sensors and chamber pressure sensors. Use of both types of sensors may provide additional data and may allow the seating system to better identify the user’s comfort level and determine what adjustments may be required to reduce high pressure and ease discomfort. In some embodiments, the cushion may also include a water-resistant or moisture-resistant barrier to protect the pressure sensors.

In some examples, the seating system may constantly monitor the pressure measurements at the plurality of pressure sensors. In some other embodiments, the one or more pressure sensors may be coupled to a processor programmed to request pressure measurements from the pressure sensors on a periodic basis (e.g., every 30 seconds or every five minutes). In some other examples, the one or more pressure sensors may be coupled to a processor programmed to request pressure measurements in response to a request from the user, caregiver, medical provider, or other interested party.

The system may then identify one or more inflatable chambers based at least in part on the pressure measurements at 504. The inflatable chambers may be associated with one or more pressure measurements that meet or exceed a threshold value. For example, the data obtained from each pressure sensor may include a pressure measurement and a pressure sensor identifier. The system may check whether the pressure measurement meets or exceeds a threshold value. If so, the system may identify one or more inflatable chambers associated with pressure sensor indicated by the pressure sensor identifier. In some examples, surface pressure sensors may be associated with a first threshold value while chamber pressure sensors may be associated with a second threshold value. In some examples, the system may identify the one or more inflatable chambers based at least in part on a medical condition associated with the user. For example, if the user is a paraplegic, the system may identify the one or more inflatable chambers based on the surface pressure sensors and may not use the chamber pressure sensors to identify the one or more inflatable chambers.

In some embodiments, the threshold value may be a maximum pressure. In some examples, the maximum pressure may be a value selected by the user’s medical provider based on the user’s medical condition. For example, if the user has hip problems, the support provided to the hips (and the corresponding pressure for the inflatable chambers supporting the hips) may vary based on the nature of those problems.

In some embodiments, the seating system may store a data structure including a correlation between pressure sensors and inflatable cushions in memory. In some examples, the data structure may be a seat map that identifies the locations of the pressure sensors and the inflatable chambers. In some other examples, the data structure may be a table that identifies one or more inflatable cushions associated with each pressure sensor identifier. For chamber pressure sensors, the table may identify the inflatable chamber in which the pressure sensor is located. For surface pressure sensors, the table may identify one or more inflatable chambers that are located underneath the pressure sensor or otherwise associated with the pressure sensor. The system may identify the one or more inflatable chambers by looking up the inflatable chambers associated with a pressure sensor identifier.

In some other embodiments, the pressures sensors may be deployed in a configuration such that a pressure map can be generated from the readings from the pressure sensors. In such a configuration, high- or low-pressure readings may be identified (e.g., measured or calculated based on the measurements) not only at the location of the pressure sensors but also at locations between the pressure sensors. The system may identify the one or more inflatable chambers based at least in part on the pressure map and a seat map. For example, the system may identify one or more locations on the cushion associated with high pressure readings based on the pressure map, and then identify one or more inflatable chambers corresponding to the one or more locations based on the seat map.

The system may then adjust the pressure for each of the one or more inflatable chambers at 506. For example, the system may decrease the pressure in each of the one or more inflatable chambers to decrease the pressure on the user.

In some embodiments, the system may further store the pressure measurements in memory. In some examples, the system may compare the pressure measurements from chamber pressure sensors to expected chamber pressures for leak detection. If a leak is detected, the seating system may provide an alert to the user, the caregiver, or another person.

The seating system may also include a gas source such as an air source. The air source may be, for example, a compressor that provides pressurized air on demand. In other embodiments, the air source may be a replaceable air tank, e.g., in the form of a cylinder that may be stored on the underside or backside of the seating system. However, other replaceable forms may also be used. In some other embodiments, the air source may be a refillable air source, e.g., a refillable cylinder that may be stored on the underside or backside of the seating system. The refillable air source may include a connection that allows it to be coupled to an external air source for refilling, e.g., when the seating system is not in use. The air source may be coupled to the cushion to supply air for use in inflating the inflatable chambers thereof.

The seating system may also include one or more valves coupled to each inflatable chamber to regulate the air inflow and outflow from that inflatable chamber. The valves may be opened to add air from the air source to increase the pressure of the inflatable chamber. The valves may also be opened to release air from the inflatable portion to decrease the pressure of the inflatable chamber. In some examples, a first valve coupled to the air source may be opened to increase the pressure and a second valve may be opened to decrease the pressure. In other examples, a single valve may be opened to either increase or decrease the pressure. The valves may be controlled (e.g., opened and closed) by a processor.

In some embodiments, at least a portion of the valve may be printed onto the inflatable chamber. For example, the inflatable chamber such as a bladder may be 3-D printed with at least a portion of one or more valves 3-D printed and integrally formed with the inflatable chamber. In some examples, the valve seat of at least one of an intake valve, an exhaust valve, and a combination intake valve and exhaust valve may be integrally formed with an inflatable chamber such as a bladder by 3-D printing the bladder.

In some embodiments, the seating system may include an actuator that controls the operation of the valves. In some examples, the valves may have a first state and a second state. In the first state, the valve may be open. In the second state, the valve may be shut. Each inflatable chamber may include at least two two-state valves, one coupled to the gas source and configured to increase the pressure in the inflatable chamber by adding gas when in the first state, and the second one not coupled to the gas source and configured to decrease the pressure in the inflatable chamber by allowing gas to escape from the chamber when in the first state. In some other examples, the valves may have a first state, a second state, and a third state. In the first state, the valve may provide gas flow in a first direction (e.g., into the inflatable chamber). In the second state, the value may provide gas flow in a second direction (e.g., out of the inflatable chamber). In the third state, the valve may be shut.

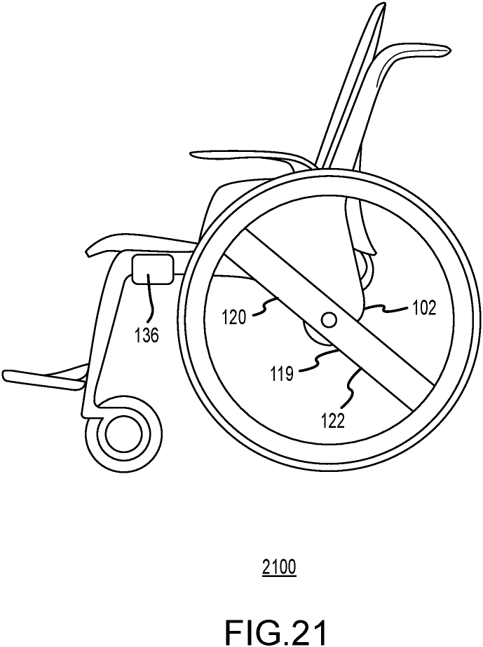

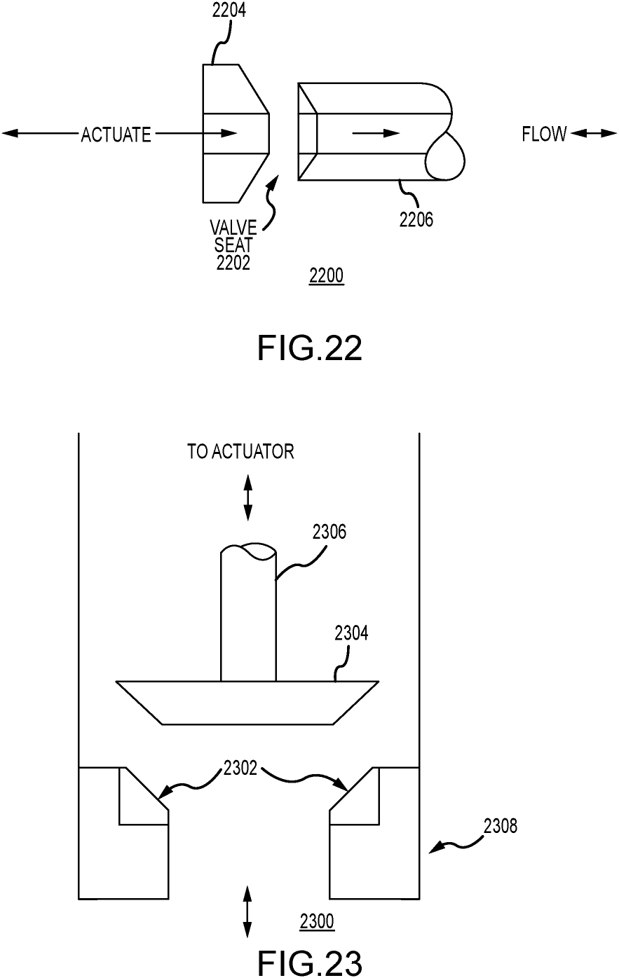

Examples of valves, actuators, and other mechanical elements that may be used in connection with the inflatable chambers are shown in FIGS. 22-25. An intake for each of the one or more inflatable chambers may be connected to a valve mechanism including one or more valves. In some examples, the valve may include a valve seat as shown in FIGS. 22 and 23. Valve 2200 includes a valve seat 2202 that may be opened and closed by an actuator head 2204. In some embodiments, the actuator head 2204 may be wider than the body of the conduit 2206 that it may be used to close. In some examples, the valve may include a poppet as shown in FIG. 23. Valve 2300 includes a valve seat 2302 that is opened and closed by a poppet 2304 controlled by an actuator which is connected to the poppet via a valve stem 2306. In some embodiments, the poppet 2304 may be narrower than the body 2308 of the conduit that it may be used to close. The poppet 2304 may include a first surface that is perpendicular to the direction of air flow into the conduit and which serves to block the air flow as well as second and third surfaces that are angled relative to the first surface and are configured to engage with the angled portions of the valve seat 2302.

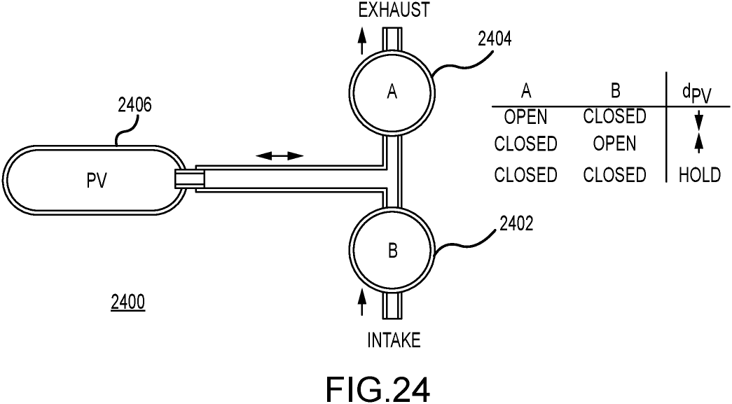

In some embodiments, the inflatable cushion arrangement may include more than one valve. For example, the inflatable cushion arrangement 2400 may include an intake valve B 2402 and an exhaust valve A 2404 as shown in FIG. 24. The intake valve and exhaust valves may be configured as shown in FIGS. 22 or 23. The one or more valves may be coupled to an inflatable chamber 2406 (labelled PV) as shown in FIG. 24. The pressure in the inflatable chamber 2406 may be increased by opening the intake valve, and the pressure may be decreased by opening the exhaust valve. When both valves are closed, the pressure will remain generally constant. The one or more valves may also be coupled to a control circuit (which may include one or more processors 404 as described with reference to FIG. 4). The control circuit(s) may individually control valves associated with each of the inflatable chambers in a cushion.

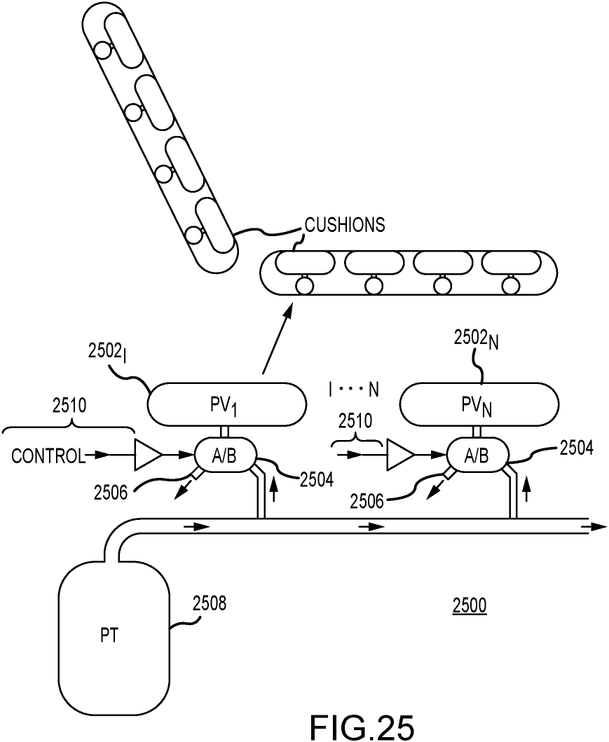

In some embodiments, an intake valve such an intake valve 2402 may be coupled to a pressure tank (PT) which may be filled with a fluid as shown in FIG. 25. For example, as illustrated by the inflatable cushion arrangement 2500 shown in FIG. 25, each inflatable chamber 2502 in a seat may have an associated intake valve 2504 and exhaust valve 2506. The intake valves 2504 may be coupled to a pressure tank 2508 which may be filled with fluid. Each intake valve 2504 may be coupled to one or more control circuits 2510 which may separately control the intake and exhaust valves so as to control the inflation of each cushion 2502 individually. Other fluid sources may alternatively be used. For example, in other embodiments the valve mechanism may be coupled to a compressor.

In some embodiments, the pressure in the inflatable chambers of the cushion may be controlled according to instructions stored in a memory of the seating system or remotely. In some examples, the pressure in the inflatable chambers may additionally or alternatively be controlled by instructions provided by a user or other person. For example, the seating system may include a control panel that allows the user or caregiver to increase or decrease the pressure in the individual inflatable portions. In other embodiments, the seating system may be controllable through communications interfaces (e.g., a Bluetooth or WiFi receiver) by a remote person such as a medical provider using, e.g., an application on a mobile phone or a computer.

In some embodiments, the processor may detect that the user has shifted based on changes in pressure across all sensors. The processor may adjust the pressures in the inflatable chambers in response to this change of position. The processor may adjust the pressures in the inflatable chambers based at least in part on historical data.

In some other embodiments, the pressure of each inflatable system may be adjusted based on feedback from the user. For example, the user or caregiver may be able to indicate that the user is feeling pain and the processor may be configured to adjust the pressure in one or more inflatable portions based on that feedback.

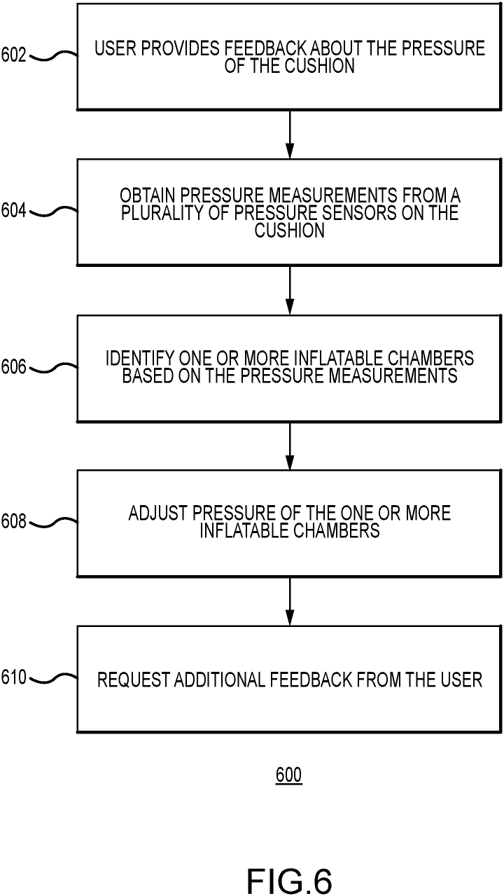

FIG. 6 is a flowchart 600 describing a method for adjusting the pressure of a cushion of a seating system in accordance with the disclosed subject matter.

The user may provide feedback about the pressure of the cushion at 602. In some examples, the user may provide the feedback through an input device on the seating system. For example, the input device may be a button or touch screen provided to allow the user to express discomfort. In some other examples, the user may provide the feedback through a software application such as a smartphone app. The software application may transmit the feedback to the seating system directly (e.g., via wireless communication) or indirectly (e.g., through a backend system). In some examples, the feedback may include an indication that the user is uncomfortable. In some other examples, the feedback may include additional information about the discomfort, such as an indication of a body part where the user is feeling discomfort or an area of the cushion where the user is feeling discomfort. For example, the user may indicate an area on a seat map corresponding to the location where the user is feeling discomfort. The seating system may receive the feedback provided by the user.

The seating system may obtain pressure measurements from a plurality of pressure sensors on and/or integrated with the cushion at 604. In some examples, the seating system may obtain pressure measurements from all of the pressure sensors on the cushion. In some other examples, the seating system may obtain pressure measurements from a subset of the pressure sensors on the cushion. For example, if the user has identified an area of the seat where they are feeling discomfort, the seating system may obtain pressure measurements only from the pressure sensors in that area of the cushion.

The seating system may identify one or more inflatable chambers at 606. The one or more inflatable chambers may be identified based on the obtained pressure measurements. For example, the seating system may identify one or more inflatable chambers associated with the highest pressure measurements of the obtained pressure measurements. In some other examples, the seating system may identify one or more inflatable chambers associated with the lowest pressure measurements of the obtained pressure measurements. In some other embodiments, if the user has identified a location associated with the discomfort, the seating system may identify an inflatable chamber associated with a highest or lowest pressure measurement in proximity to the identified location (e.g., within about one inch, within about two inches, or within about three inches of the identified location). In some embodiments, if the user has identified a body part associated with the discomfort, the seating system may identify an inflatable chamber based in part on the body part. For example, if the user identifies hip pain or numbness, the seating system may store information about the locations on a pressure map that are most likely to cause hip pain or numbness and may identify one or more inflatable chambers near those locations.

In some embodiments, the seating system may identify the one or more inflatable sensors based on stored pressure settings. For example, the seating system may store one or more profiles that specify pressure values for each of the inflatable chambers. In some examples, the one or more profiles may include a first profile (e.g., associated with a firm cushion) and a second profile (e.g., associated with a soft cushion). The user may identify one of the known settings in providing feedback at 602. The seating system may compare the pressure values associated with the selected profile with the pressure values measured by the chamber pressure sensors to identify one or more inflatable chambers that require an increase in pressure and/or one or more inflatable chambers that require a decrease in pressure.

In some embodiments, the seating system may determine a recommended profile based at least in part on user information. The user information may be information entered by a user such as height and weight. In some embodiments, the user information may additionally or alternatively include body measurements captured by a camera or video camera on the seating system. The seating system may determine recommended pressure values for each inflatable chamber based at least in part on the user information. The seating system may then compare the recommended pressure values with the pressure values measured by the chamber pressure sensors to identify one or more inflatable chambers that require an increase in pressure and/or one or more inflatable chambers that require a decrease in pressure.

In some embodiments, the seating system may then adjust the pressure of the one or more inflatable chambers at 608. In some examples, the seating system may increase the pressure in the one or more inflatable chambers by adding more fluid to the inflatable chambers. In some other examples, the seating system may decrease the pressure in the one or more inflatable chambers by allowing fluid to escape from the one or more inflatable chambers.

In some embodiments, the seating system may then request additional feedback from the user at 610. For example, the seating system may request user feedback on whether the user is still uncomfortable. The user may indicate that they are no longer in discomfort, in which case the process ends. Alternatively, the user may indicate that they are still in discomfort, in which case the process may return to step 604 and further adjustments to the pressure of the cushion may be made. The seating system may also request user feedback on whether the user is more comfortable or less comfortable than before the adjustments were made. If the user indicates they are less comfortable, the seating system may reverse the pressure adjustments before returning to step 604. If the user indicates they are more comfortable, the seating system may continue to make further pressure adjustments on top of the initial pressure adjustments. The process may continue until the user indicates they are comfortable.

In some embodiments, the pressure of each inflatable portion may be varied in time to provide a massage functionality. By controlling the variance of pressure in each inflatable portion, the seating system may provide an undulation. The frequency of the undulation may be programmed to a certain rate or may be selected randomly or semi-randomly, and in some cases may be varied by the user, caregiver, or medical provider. This functionality may also be activated and deactivated at programmed, random, or semi-random intervals, or activated on demand by the user, caregiver, or medical provider. This feature may be provided and/or implemented for comfort and/or medical reasons, such as providing pain relief.

In some embodiments, the seating system may also adjust the pressure in the inflatable chambers in accordance with an operating mode. For example, when the user is attempting to get out of the seating system, either on their own or with the assistance of another such as a caregiver, the seating system may provide a higher pressure on one side of the cushion (e.g., the back) and a lower pressure at the other side of the cushion (e.g., the back) to make it easier for the user to slide out of the chair by providing a downward slope or make it easier for the caregiver to grasp the user and assist them out of the chair. In other examples, such as where the Inflatable chambers are formed in a concentric circle pattern, the seating system may provide a higher pressure in the center of the cushion and a lower pressure on the outer portions of the cushion. When the user is attempting to get back into the seating system, the pressure gradient may be provided in the opposite direction. In still other examples, the pressures may be varied to accommodate mechanical or motorized lift systems that caregivers utilize to place or extract a user from a seating system.

The seating system in accordance with the disclosed subject matter may additionally or alternatively include other methods for adjusting the operation of the seating system based on sensor readings. For example, the seating system may also include a heating device. The processor may activate the heating device when a temperature sensor reading falls below a certain threshold to provide warmth to the user, or at the request of the user. In some embodiments, the seating system may also include a cooling unit which may be activated by the processor when a temperature sensor reading exceeds a certain threshold, or at the request of the user. In some examples, the heating and/or cooling devices can be manually overridden and turned on or off by the user or the caregiver. The heating and cooling elements may have associated timers to limit the application of heating and cooling systems. The seating system may also be configured to deactivate the heating system, cooling system, and other systems of the seating system when the seating system detects that the user is no longer in the seating system, e.g., when a weight detected by a weight sensor drops below a seated weight threshold and/or when a pressure detected by the pressure sensor drops below a seated pressure threshold.

In some embodiments, the heating and/or cooling device may be integrated with a cushion. For example, the seating system may include heating elements to warm the air that is located in or is to be injected into the inflatable portions. In other examples, the seating system may include cooling elements (such as a heat exchange) to cool the air. A thermal exchange device may be stored on the underside of the seating system (e.g., in a chassis under the seat of a wheelchair).

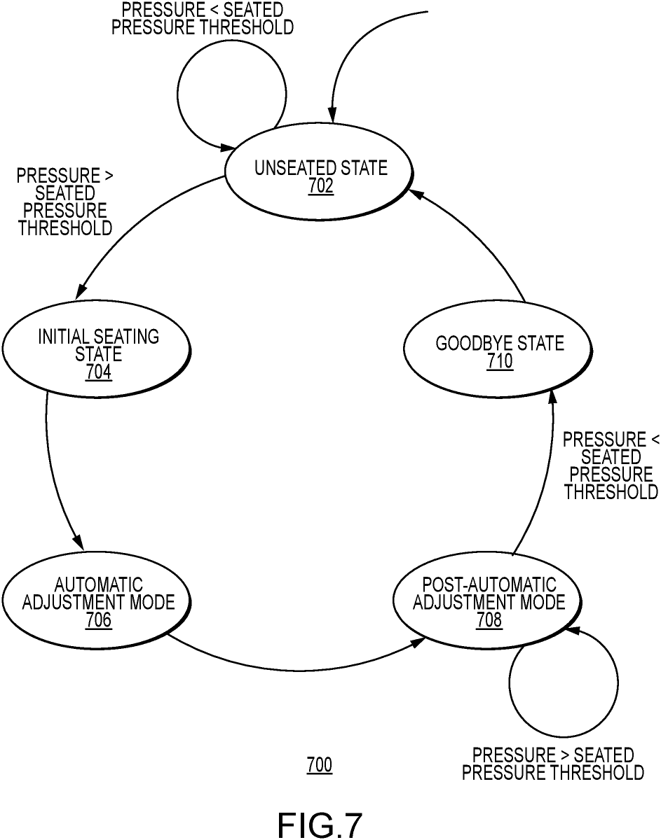

FIG. 7 is a state diagram 700 for adjusting the pressure of a cushion on a seating system in accordance with the disclosed subject matter.

The first state 702 may be an unseated state corresponding to a time period before the user sits in the seating system. For example, the seating system may enter the first state 702 as soon as the seating system is turned on. The seating system may remain in the first state 702 as long as one or more thresholds is not exceeded. For example, in some embodiments the seating system may remain in the first state until the pressure measurements from a plurality of pressure sensors on a cushion meet or exceed a seated pressure threshold value. In some other embodiments, the seating system may remain in the first state until the weight measurements associated with a weight sensor meet or exceed a seated weight value.

When the user sits in the seating system, the seating system will transition from the first state 702 to a second state 704. This transition may occur when the pressure measurements and/or weight measurements meet or exceed a threshold value as discussed above. In some examples, when the user enters the second state 704, the seating system may invite the user to adjust the cushion. For example, the seating system may display an automatic adjustment button on a display screen of the seating system and/or may cause a software application on a computing device to send the user and/or the caregiver an alert inviting them to enter the software application and start the adjustment process.

The seating system may then transition from the second state 704 to a third state 706. In some embodiments, the seating system may automatically transition from the second state 704 to the third state 706 after a set period of time, e.g., thirty seconds after the seating system enters the second state 704. In some other embodiments, the seating system may transition from the second state 704 to the third state 706 based on user input, e.g., when the user and/or caregiver selects the automatic adjustment button on the display screen of the seating system or through the software application.

Upon entering the third state 706, the seating system will automatically adjust the pressure of one or more inflatable chambers in the cushion. In some examples, the seating system may adjust the pressure of one or more inflatable chambers based on a profile specifying a pressure value for each user. In some embodiments, the seating system may adjust the pressure of one or more inflatable chambers based on a recommended profile specifying recommended pressure values for each user. In some examples, the recommended profile may be determined based on user information such as height, weight, and body measurements. In some other examples, the recommended profile may be determined based on historical pressure data.

After completing the automatic adjustment of the pressure for one or more inflatable chambers, the seating system may enter a fourth state 708. In the fourth state 708, the seating system may continue adjusting the pressure values for one or more inflatable chambers based on, e.g., pressure readings and user input. In some examples, the seating system may automatically adjust the pressure of the cushion as described above with reference to FIG. 5 when in the fourth state 708. In some examples, the seating system may adjust the pressure of the cushion based on user input as described above with reference to FIG. 6 when in the fourth state 708. In some examples, the seating system may adjust the pressure based on learned preferences, historical pressure data, and/or user behavior when in the fourth state 708.

The seating system may remain in the fourth state 708 as long as the weight and/or pressure measurements meet or exceed seated threshold values. When the weight and/or pressure measurements drop below the seated threshold values, the seating system may transition from the fourth state 708 to the fifth state 710. In some examples, the seating system will remain in the fourth state 708 until pressure measurements from all of a plurality of pressure sensors drop below the seated threshold value. In some examples, the seating system may remain in the fourth state 708 for a period of time after the weight and/or pressure measurements drop below the seated threshold to avoid moving to the fifth state 710 when the user is only briefly out of the seating system (e.g., when the user is repositioning or being repositioned). In some examples, the seating system may store pressure sensor data for a session when the seating system enters the fifth state 710. In some other examples, the seating system and/or software application may issue a goodbye message when the seating system enters the fifth state 710. The seating system may then transition from the fifth state 710 back to the first state 702 to await the user’s return.

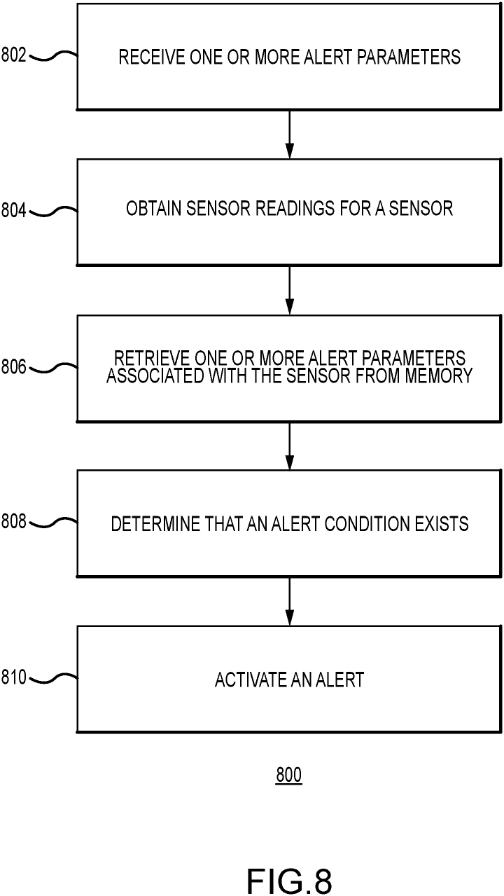

In some embodiments of the disclosed subject matter, the seating system may be configured to monitor the health of the user and/or the operation of the seating system, and to provide an alert when an unsafe condition is detected. FIG. 8 is a flowchart 800 describing a method for activating an alert in accordance with embodiments of the disclosed subject matter.

One or more alert parameters may be specified at 802. The alert parameters may be specific to a sensor. In some examples, the alert parameters may include a maximum value. For example, a maximum value for a moisture detector may be set such that any amount of moisture exceeding that value will trigger an alert. In some examples, the alert parameters may include a minimum value such as a minimum blood glucose level such that a blood glucose reading below that amount will trigger an alert. In some other examples, the alert parameters may specify a range including a lower limit and an upper limit. The range may specify an acceptable range for the sensor readings such that any value outside of that range will trigger an alert. For example, the alert parameters may identify an acceptable blood oxygen range, an acceptable heart rate range, or an acceptable temperature range.

In some embodiments, alert parameters may be set for two or more levels of alerts. For example, the alert parameters for a blood glucose sensor may identify a first blood glucose minimum associated with a first level alert and a second blood glucose minimum associated with a second level alert.

In some embodiments, alert parameters may be set based on input from the medical provider. For example, the medical provider may provide alert parameters corresponding to a user’s medical condition.

The system may then obtain sensor readings for a sensor at 804. The sensor may be, for example, blood oxygen sensor such as a pulse oximeter, a heart rate sensor, a temperature sensor, a blood glucose sensor, a blood pressure sensor, a weight sensor, a speed sensor, a pressure sensor, an orientation sensor, or other sensors as known in the art. One or more processors (such as the one or more processors 404 as described in connection with FIG. 4) may receive the sensor readings from the sensor through wired or wireless communication.

The processors may then retrieve the one or more alert parameters associated with the sensor from memory at 806. The one or more alert conditions may be stored in internal memory 406a or external memory 406b as described above in connection with FIG. 4.

The processors may then determine whether an alert condition exists at 808. The processors may determine whether an alert condition exists based on the sensor reading and the retrieved alert parameters. For example, if the alert parameters identify an acceptable range, the processor may determine that an alert condition exists when the sensor reading is outside of the acceptable range.

If the processors determines that an alert condition exists, the processor activates an alert at 810. In some embodiments, the processors activate a visual indicator when an alert condition exists. For example, the processors may illuminate a warning light when an alert condition exists. In some other examples, the processors may change the color of a warning light (e.g., from green to red) when an alert condition exists. In some embodiments, the processor may activate an audio alert such as providing a voice message or other audio indication through a speaker. In some other examples, the processor may cause a text message to be sent to a local or remote display device concerning the alert. In some examples, the text message may include details about the alert including the type of alert (e.g., high heart rate or low heart rate) and the sensor reading (e.g., 60 bpm). In some examples, the text message may also provide suggested actions (e.g., an instruction to call the user’s medical provider).

In some embodiments, the processors may transmit a message to an electronic medical records system and/or a software application associated with the user’s medical provider. In some examples, the processors may transmit the message to the medical provider only when parameters set by the medical provider are satisfied. In some examples, the processors may transmit a text message to one or more cell phones specified by a user, such as a cell phone associated with the caregiver, a cell phone associated with a medical provider, or a cell phone associated with a family member such as the user’s parent, spouse, or child.

Additional information about the alert may be provided based on a request from the user, caregiver, medical provider, or other interested party. For example, upon noticing the alert, a person may send a request for additional information about the alert to the seating system (e.g., through an application on the person’s mobile device or a display screen integrated with the seating system). In some examples, the person may also send a request for an updated sensor reading (e.g., because the user or caregiver has adjusted the sensor and wants to check whether the initial sensor reading was correct). The seating system may receive the request, send a response including details about the alert, request an updated sensor reading from the sensor, and send a response including the updated sensor data.

In some embodiments, the processor may adjust the operation of the seating system if an alert condition is detected. For example, if the processor determines that the user’s temperature is above a threshold value, the processor may activate the cooling system to provide relief to the user.

The processor may also store the sensor readings. In some examples, the processor may store the sensor readings by writing the data to memory. In some examples, the processor may store the sensor readings only when an alert condition is detected.

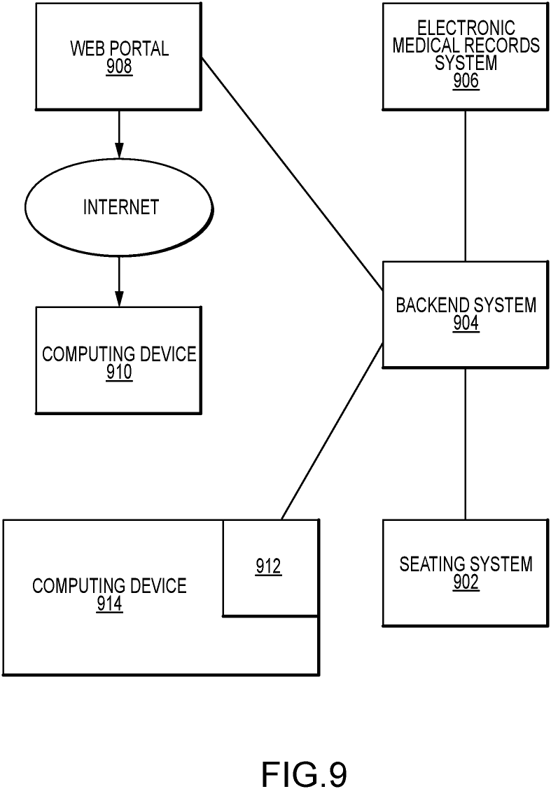

FIG. 9 is a block diagram of a networked system 900 in accordance with embodiments of the disclosed subject matter. The system 900 may include a seating system 902, a backend system 904, and an electronic medical records system 906. The seating system 902 may be, for example, a wheelchair such as wheelchair 100 as described in connection with FIG. 1.

The seating system 902 may communicate with the backend system 904 through one or more communication units or communication ports. The backend system 904 may include one or more transceivers that allow the backend system 904 to receive information from and send information to the seating system 902. The backend system 904 may also include one or more memories configured to store data received from the seating system 902. For example, the backend system 904 may include one or more computers and/or one or more servers. In some examples, the one or more servers may include one or more cloud servers. In some examples, the backend system 904 may be a distributed system.

The seating system 902 may include one or more sensors. In some examples, the sensors may be biometric sensors. For example, the one or more sensors may include a heart rate sensor, a blood pressure sensor, a blood glucose sensor, a blood oxygen sensor such as a pulse oximeter, a temperature sensor, a pressure sensor, a moisture sensor, or any other sensors as known in the art. The seating system 902 may be programmed to transmit sensor data from any of the sensors to the backend system 904 for storage. In some embodiments, the seating system 902 may be configured to transmit the sensor data to the backend system 904 in real-time. In some examples, the seating system 902 may be programmed to transmit the sensor data to the backend system 904 at periodic intervals. In further embodiments, the seating system 702 may be configured to transmit the sensor data to the backend system 904 upon the occurrence of a particular event. For example, the seating system 902 may be configured to transmit sensor data to the remote data store if an alert condition is detected (e.g., the user’s blood oxygen levels are outside of an acceptable blood oxygen range). As another example, the seating system 902 may be configured to transmit sensor data to backend system 802 in response to a request, e.g., from a medical provider or the backend system 904. As a further example, the seating system 902 may be configured to transmit sensor data when the seating system 902 is coupled to an electrical source (e.g., when the seating system 902 is plugged in at the end of the day) to conserve power (which may be desirable where only safe conditions are detected). Such a configuration may be particularly desirable where the data connection is wired. In some examples, the seating system 902 may be configured to transmit sensor data upon detecting that a communication channel has been re-established if, for example, the seating system 902 is unable to communicate with the backend system 904 for a period of time. In some embodiments, the user, caregiver, or medical provider may be offered the option of selecting how the seating system 902 will communicate the sensor data to the backend system 904. The transmission of data to the backend system 904 store may be wired or wireless.

In some embodiments, sensor data may be communicated and stored in an encrypted format. For example, the seating system 902 may include an encryption unit configured to encrypt the sensor data before it is transmitted to the backend system 904. The seating system 902 may also include a decryption unit configured to decrypt sensor data received from the backend system 904 (e.g., historical sensor data that may be requested by the user or caregiver). The backend system 904 may include similar encryption and decryption units. In embodiments of the disclosed subject matter, the sensor data may be stored in a format that is compliant with applicable regulations covering the protection of personal information such as, for example, regulations associated with HIPAA (the Health Insurance Portability and Accountability Act) in the United States and/or GDPR (General Data Protection Regulation) in Europe. In some examples, non-sensitive information may be transmitted and stored without such encryption and formatting precautions. For example, information about the speed and orientation of the seating system may not require the same protection as information about the user’s heart rate and blood oxygen levels.

In some embodiments, the backend system 904 may also store additional information associated with the user. For example, the backend system 904 may store an account identifier associated with the user. In some other examples, the backend system 904 may store an identification of the user’s medical provider or other persons who are authorized to view the user’s information (e.g., the sensor data). In some examples, the backend system 904 may also include an identification of the types of data each authorized user may view. For example, while a user’s medical provider may be granted access to all of the user’s sensor data, the user may want a family member to have access to only limited information (e.g., the GPS location of the seating system, the user’s heart rate, and/or the user’s blood glucose level). The backend system 904 may also store account information for each authorized user, which may include a user identifier and a password.

In some embodiments, the backend system 904 may be configured to communicate with one or more electronic medical records systems 806. The electronic medical records system 906 may be an electronic medical records system used by the user’s medical provider. In some examples, the electronic medical records system 906 may be an electronic medical records system used by another medical provider such as a hospital that is treating the user in an emergency situation. The backend system 904 may authenticate the electronic medical records system 906 using techniques as known in the art. By accessing the sensor data through the electronic medical records system 906, a treating medical provider may be able to better treat the user (e.g., by understanding the user’s medical condition during a period before, during, and after an emergency medical event such as a heart attack or stroke).

In some embodiments, the electronic medical records system 906 may also transmit information to the backend system 904 and/or the seating system 902. For example, the user’s medical provider may enter one or more alert parameters into the electronic medical records system 906. The medical provider may select the alert parameters based at least in part on the sensor data and/or the user’s medical history. The electronic medical records system 906 may transmit the alert parameters to the backend system 904, which may store the alert parameters in memory and transmit the alert parameters to the seating system 902. In some other embodiments of the disclosed subject matter, the electronic medical records system 906 may be configured to communicate directly with the seating system 902. In some other examples, the electronic medical system 806 may transmit a treatment plan for the user. The medical provider may develop the treatment plan based at least in part on the sensor data received from the seating system 902 or the backend system 904, including the historical sensor data stored at the backend system 904. The treatment plan may be made available to the user at the seating system 902 (e.g., via a display screen of the seating system 902) or via a computing device including a software application for accessing the treatment plan.

In some embodiments, the backend system 904 may make sensor data available to the user, the caregiver, the medical provider, or other persons designated by the user (e.g., non-caregiver family members). In some examples, the information may be accessible through existing electronic medical record channels as discussed above.

In some embodiments, the information may be available through the Internet. For example, the system 900 may include a web portal 908 coupled to the backend system 904. The user, caregiver, medical provider, or others may access a website associated with the web portal 908 by entering the appropriate address (i.e., URL) on a computing device 910 connected to the Internet 812. The web portal 908 may send a request for a user identifier and password to the computing device 910 (e.g., as part of the HTML or other code for the website). The web portal 908 may provide sensor data to the computing device 910 only if the user identifier and password match the user identifier and password of a user authorized to view the sensor data.

In some embodiments, the information may be available through a software application such as an application 912 running on a computing device 914. The software application 912 may be, for example, a desktop application or a mobile application. The computing device 914 may be, for example, a mobile phone or tablet device. The user, caregiver, medical provider, or other person may need to input a user identifier and password to access the user’s sensor data through the software application 912.

In some embodiments, one or more persons designated by the user may be provided with or given the option of receiving reports related to the user’s sensor data, such as periodic (e.g., daily or weekly) reports and/or alerts, through the web portal 908 or the software application 912. For example, the backend system 904 may generate a report based on the sensor data received from the seating system 902. The report may be generated based on the sensor data for a predetermined time period, such as the last day or the last seven days. In some examples, each sensor measurement may be tagged with a time indicator that indicates when the measurement was taken. The backend system 904 may transmit the report to the electronic medical records system 906 associated with the user’s medical provider. The backend system 904 may also make the report available through the web portal 908 and the software application 912, e.g., in response to a request to access the report. Similarly, an alert may be sent to the electronic medical records system 906, via the webs portal 908, and/or through the software application 912 when the user’s sensor data indicates an alert condition, as discussed in greater detail in connection with FIG. 8 above.

In some embodiments, the seating system may be controlled based on commands provided by a caregiver or medical provider, e.g., through the software application 912. For example, the caregiver may use the mobile application 912 to cause the seating system to stop moving (e.g., by shutting off a motor or other propulsion system, or applying an electronic brake). As another example, a medical provider may request one or more sensors to perform another reading or perform self-diagnostics to double-check a reading or confirm that the sensor is properly operating. The medical provider’s request may be transmitted to the seating system 902 via the electronic medical records system 906, the web portal 908, or the software application 912.

In some embodiments, the user, caregiver, or medical provider may configure the seating system through the software application 912 or by using another computing device (e.g., computing device 910).

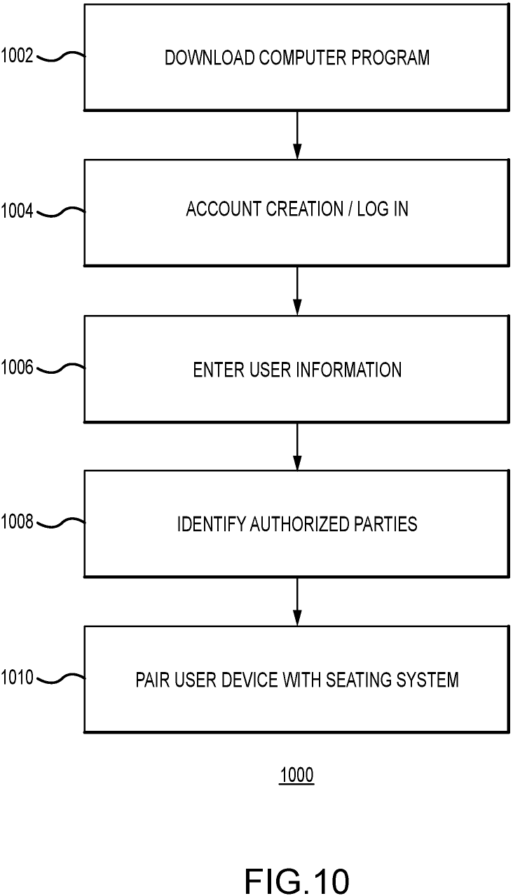

FIG. 10 is a flowchart 1000 showing the steps for setting up a seating system in accordance with some embodiments of the disclosed subject matter. The user or a caregiver, family member, or other person may download a computer program (e.g., a mobile application) to a user device such as a mobile phone or tablet device at 1002. In some examples, the person may scan a QR code provided with the seating system to download a mobile application. In some other examples, the person may download a mobile application through an application store. In some other examples, the person may download a desktop application over the Internet. In embodiments where the seating system is set up through a website, the person may not need to download any software. In some other examples, the computer program may be pre-stored in a memory of the seating system and the person may access the program through a display screen or other input/output device on the seating system.

The person may then create a new account or log into an existing account at 904. The person may create a new user identifier and password when setting up the account or may enter an existing user identifier and password for the existing account.

The person may then enter user information at 1006. The user information may include basic information such as name, age, gender, and the like. In some examples, the person may identify one or more medical conditions or clinical diagnoses associated with the user. For example, the person may be allowed to identify one or more conditions from the following exemplary list: paraplegic, broken limb, quadriplegic, muscular dystrophy, disorientation, and/or neurological. In some other examples, the person may be prompted to indicate the medical conditions or clinical diagnoses, e.g., by entering established diagnostic codes. In some examples, the medical condition information indicated by the person may be used for operation of the wheelchair, e.g., to determine adjustments to the inflation of the cushion and/or to determine emergency conditions associated with one or more of the sensors integrated with the seating system. In some examples, the person may identify additional user information such as age or birthdate, height, and/or weight. In some examples, the user information may be used to provide recommended settings or automatically configure the settings of the seating system. In some examples, the person may also identify a user activity level. The identified activity level may be used in operation of the seating system, e.g., in determining how to adjust the inflation of the cushion. In some examples, the person or another person may subsequently modify information previously entered by the person.

The person may also identify one or more caregivers or other persons who may be given access to information from the seating system at 1008. For example, the person may enter a phone number or email address associated with a caregiver to be given access to the seating system, which may cause a backend system (such as backend system 904 as described with reference to FIG. 9) to send an email, text message, or other communication to the caregiver inviting them to create an account and/or accept the invitation to access information about the user’s seating system. In some examples, the person may also identify a level of access for each identified caregiver. For example, the person may allow a family member that does not provide caregiving functions to view sensor data from the seating system but not control the operation of the seating system. In some examples, the person may allow the family member access to only certain types of sensor data. The person may allow a caregiver to view the sensor data and control the operation of the seating system (e.g., to inflate or deflate the cushion). In some examples, the person may select one or more persons who are allowed to view the location of the seating system but not to view any of the sensor data or control the operation of the seating system, which may be helpful if the user cannot be located. The person may also identify one or more medical or other care providers, who by default may be given access to all sensor data from the seating system to assist in monitoring the patient’s health. The person may change the default settings to limit the medical or other care provider’s access to data from the seating system.

The person’s device may then pair with the seating system via wireless communication at 1010. For example, a person’s device may include a transceiver such as a Bluetooth transceiver that may communicate with a corresponding transceiver of the seating system. In some examples, the person may need to enter some information about the seating system (e.g., an identification number printed on the seating system) to pair the seating system with the device. The seating system may thereafter send information to the person’s device or another paired device (such as a caregiver’s device, which may be paired in a similar manner).

In some embodiments, the user, the user’s medical provider, and the user’s authorized caregivers or other persons may also be permitted to access information from the seating system through a computer program or website that communicates with the backend system. In such embodiments, no pairing may be required.

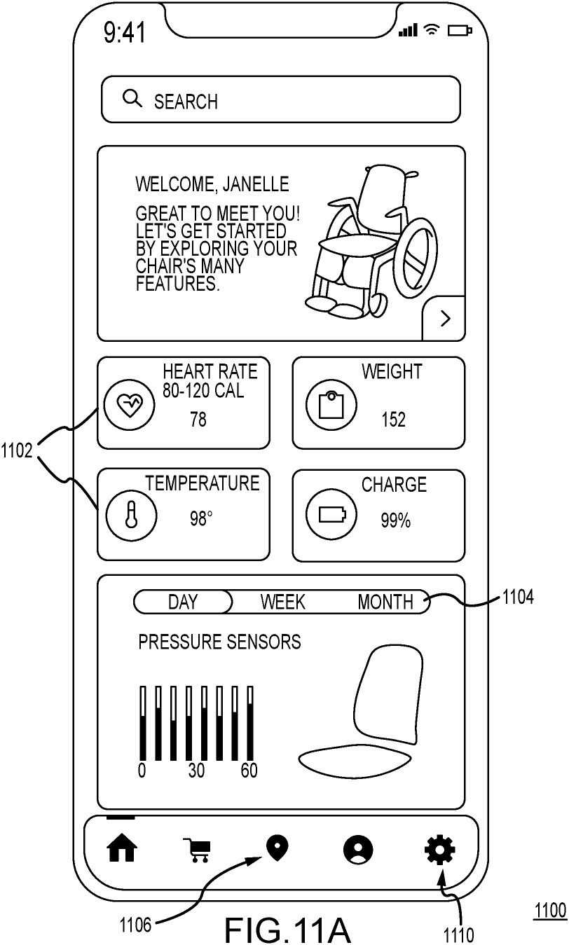

FIGS. 11A and 11B show examples of the information that may be provider to a user, caregiver, medical provider, or others via a software application such as software application 912 described with reference to FIG. 9 in accordance with embodiments of the disclosed subject matter. The information may include, for example, current sensor readings 1102. The current sensor readings 1102 may include a current heart rate, a current weight, a current temperature, and a current charge for the seating device. The current sensor readings 1102 also include current pressure sensor readings. The current sensor readings 1102 may also include current readings from any of the other sensors of a seating system as described herein. The current sensor readings 1102 may be received via a direct connection (e.g., a wireless connection) between the seating system and the computing device on which the software application is running. In some other examples, the current sensor readings 1102 may be transmitted from the seating system to the computing device via a backend system.

In some embodiments, the information may also include historical sensor readings 1104. In some examples, the historical sensor readings 1104 may include historical sensor readings for one or more intervals, such as sensor readings for the past day, sensor readings for the past week, and/or sensor readings for the past month. In some examples, the historical sensor readings 1104 may be accessible by a user selecting a sensor. For example, the user may be able to access historical sensor readings 1104a for a weight sensor by selecting the tile 1102a showing the current sensor reading for the weight sensor. Selection of the sensor shown in FIG. 11A may cause the screen to change to display the more detailed information about the sensor as shown in FIG. 11B. In some examples, the seating system may not store historical data readings for the full time period. The software application may therefore need to retrieve historical sensor readings from a backend system even if the software application is in direct contact with the seating system. The data may be displayed in alphanumeric format, in graphical format, or in both alphanumeric format and graphical format.

In some embodiments, the user also may be able to access location information for the seating system through the software application. For example, the user may be able to access location information by selecting an icon 1106 associated with the location information.

In some embodiments, the user may also be able to access user profile information and settings for the seating system through the software application. For example, the user may access the user profile by selecting the icon 1108 associated with the user profile. The user may be able to add or modify user information, caregiver information, or other information as discussed with reference to FIG. 10. As another example, the user may access settings for the seating system by selecting the icon 1110 associated with the settings. The user may be able to modify operational parameters associated with the seating system through the software application. For example, the user may be able to set a minimum temperature and a maximum temperate. This information may be transmitted to the seating system. If the temperature measured at the seating system drops below the minimum temperature, the seating system may activate a heating device to bring the temperature back to the minimum temperate. Similarly, if the temperature measured at the seating system drops below the maximum temperature, the seating system may activate a cooling device to bring the temperature back to a maximum temperature.

In some embodiments, other information may be available through the software application. For example, the user and/or caregiver may be able to access a treatment plan for the user through the software application.

The information available through the software application as discussed with reference to FIG. 11 may alternatively be accessed through a website (via a web portal such as web portal 908 as discussed with reference to FIG. 9) or at the seating system (e.g., at a display screen attached to the seating system).

In some embodiments, the seating system or a software application (e.g., the user’s or caregiver’s mobile application) may be configured to communicate with a medical provider computer system (which may be an electronic medical record system such as electronic medical records system 906 as described with reference to FIG. 9). In some examples, the seating system or software application may communicate with the medical provider computer system to facilitate the provision of medical care to the user. In some examples, a medical provider computer system may receive information (e.g., sensor information) from the seating system and/or software application and may also access medical information from other medical record databases (which may be a part of the electronic medical records system 906). This may allow the medical provider to view information from the seating system (such as sensor information) as well as other medical information such as, for example, lab results, prescriptions, care plans, and the like. This may allow the medical provider to have more complete information to evaluate the information received from the seating system and/or the user’s overall health.

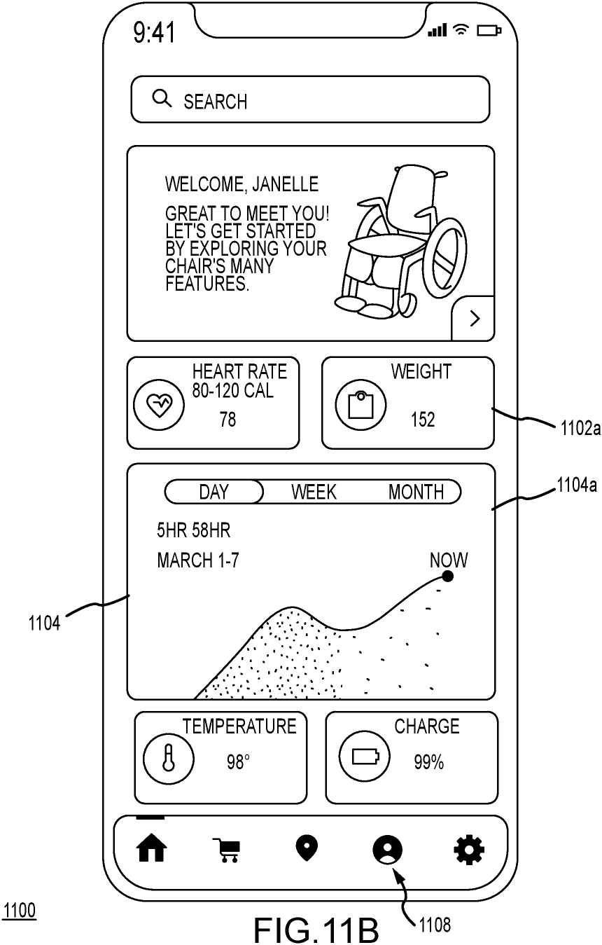

FIG. 12 is a flowchart describing a method for communicating with a medical or other care provider computer system in accordance with some embodiments of the disclosed subject matter.

The user may schedule an appointment with a medical or other care provider at 1202. In some examples, the user may schedule the appointment through a software application such as software application 912 as described with reference to FIG. 9. In some examples, the user may schedule the appointment through the seating system, e.g., using a display screen attached to the seating system. In some examples, the user may schedule the appointment through other communications channels, e.g., in-person (such as at the end of a previous appointment) or via telephone. The medical provider computing device may send a confirmation to the software application. For example, an electronic medical record system (such as electronic medical records system 906) may transmit a confirmation to a software application (such as software application 912), either directly or via a backend system (such as backend system 904). The appointment information may be stored in the device on which the software application is running, in a memory of the seating system, or on a backend system. The software application, seating system, or backend system may be configured to provide reminders to the user and/or caregiver as the appointment approaches.

The user may be invited to check in for the appointment at 1204. In some examples, the seating system or software application on a computing device may detect that the user is approaching the office of the medical provider, e.g., using a location tracker such as a GPS system in the seating system or user device, and may send a notification to the user inviting the user to check in for the appointment. For example, the notification may be provided to the user when the seating system or user device is determined to be within half a mile of the medical provider office. In some examples, the notification may be provided only if the current time is within a certain amount of time before the scheduled appointment, e.g., half an hour.

The user may then complete the check-in process at 1206. In some examples, the user may complete the check-in process through the user device or seating system, which may minimize the amount of time the user spends in a waiting room with other patients that may be ill, which in turn may minimize the risk that the user becomes ill when visiting the medical provider office.

The check-in information may then be transmitted to the medical provider computer system at 1208. The check-in information may be transmitted to the medical provider directly from the user device or seating system or indirectly, e.g., through the backend system. The provider medical office may use that information to complete the registration process for the patient.

As part of the registration process, the medical provider computer system may request the sensor data from the seating system at 1210. In some examples, the medical provider computer system may request historical sensor data stored at the backend system and/or in a memory of the seating system. In some other examples, the medical provider computer system may additionally or alternatively request current sensor data from the seating system. The sensor data may be provided to the medical provider for use during the user’s appointment.

The medical provider computer system may notify the medical provider is ready to begin the appointment at 1212. In some examples, the medical provider system may determine when an exam room is available for the user. For example, the medical provider computer system may receive input from a member of the medical provider’s office indicating that an exam room is available. The medical provider computing system may transmit a notification to the user device and/or seating system indicating that the exam room is available, further limiting the amount of time the user may be required to wait in a waiting room.

The user may thereafter enter the exam room for the appointment at 1214. A medical provider (e.g., a doctor or nurse) may examine the user and review sensor data in determining the appropriate course of treatment. In some cases, the medical or other care provider (such as a seating specialist, physical therapist, or occupational therapist) may request additional sensor data via the medical provider computer system, control the seating system (e.g., adjust the inflation of the cushion) via the medical or other care provider computer system, or update alert parameters associated with one or more of the sensors of the seating system via the medical or other care provider computer system.

Following the appointment, the medical provider computer system may provide a notification that the appointment has ended to the seating system and/or user device at 1216. In some examples, the medical provider computer system may also provide additional information (e.g., care recommendations) to the user through the user device. In some examples, the user may also be invited to schedule a next appointment with the medical provider through the seating system and/or mobile device, thereby further limiting the amount of time the user needs to spend in the waiting room or other common areas of the medical provider office.

In some embodiments, the medical provider computer system may communicate with a device associated with an authorized caregiver or other authorized person as an alternative or in addition to communicating with the seating system and/or user device.

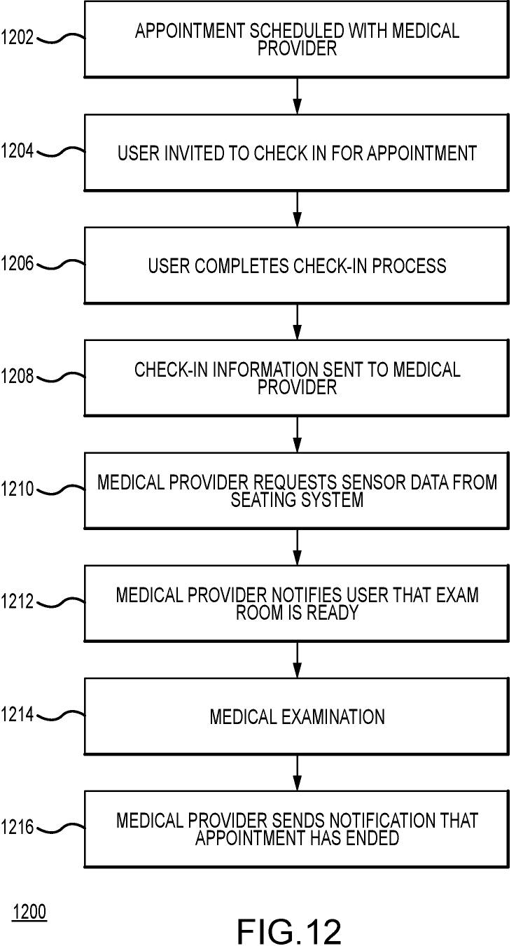

FIG. 13 is a block diagram of an armrest 1300 of a seating system in accordance with embodiments of the disclosed subject matter.

The armrest 1300 may include a display screen 1302. In some other examples, the display screen 1302 may be located in other locations on the seating system such as behind the back portion so that it is viewable to the caregiver rather than the user or offset from the armrest and viewable by the user and other persons. The display screen 1302 may be configured to display information for the user. In some examples, the display screen 1302 may have a touchscreen and may also be configured to receive input from the user. The display screen 1302 may display any information discussed herein to the user. For example, in some examples the display screen 1302 may display sensor measurements. In some examples, the display screen 1302 may display text alerts when an alert condition is detected. In some examples, the display screen 1302 may display an icon requesting the user to begin an automatic cushion adjustment. In some examples, the display screen 1302 may display an icon for the user to select when they are uncomfortable and would like to request a seat adjustment.

In some embodiments, the display screen 1302 may display instructions for a user. In some examples, the instructions may be based on a medical condition associated with the user. For example, if the user is a diabetic, they may be required to take blood glucose measurements on a periodic basis. The seating system may be configured to display a reminder on the display screen 1302 when it is time for the user to measure their blood sugar. In some other examples, the seating system may be configured to display a reminder for the user to take medicine according to a medicine schedule on the display screen 1302.

The armrest 1300 may also include one or more communication interfaces 1304. The communication interfaces 1304 may be USB ports. In some examples, the seating system may include one or more sensors that are removably attachable to the seating system via communication interfaces 1304. The one or more removable sensors may be stored in a storage location (such as a flexible pack, box. or drawer) located underneath the seat portion of the seating system or behind the seat back when not in use. The one or more removable sensors may be, for example, a pulse oximeter configured to transmit blood oxygen readings to the memory of the seating system via the communication interfaces. In some other examples, the communication interfaces 1304 may be located on other places on the seating system, such as on the side of the armrest 1300 or on a front or side of the seat portion.

The armrest 1300 may include one or more input buttons 1306. In some examples, the input buttons 1306 may be a general button, e.g., it may be pushed to activate a variety of functions. For example, the armrest 1300 may include a first input button 1306 and a second input button 1306 that may be used to respond yes and no, respectively, to a question presented to the user (e.g., through the display screen 1302). In some other examples, each of the one or more input buttons may be specialized. For example, a first input button 1306 may be used only to request adjustments to the pressure of the cushion of the seating system, a second input button 1306 may be used to adjust the incline of the seat portion of the seating system, and a third input button 1306 may be used to activate the massage functionality of the cushion.

The armrest 1300 may include one or more indicators 1308, which may be indicator lights. For example, a first indicator light 1308 could correspond to the power state of the seating system. The first indicator light 1308 may be illuminated in green when the seating system is charged above a threshold charge (e.g., 20% battery) and may be illuminated in red when the system charge drops below the threshold charge. In some other examples, one or more of the indicator lights 1308 may indicate an alert condition exists when illuminated. For example, one or more of the indicator lights 1308 may be illuminated when the seating system measures a moisture level above a moisture threshold, or a heart rate above a heart rate threshold.

In some embodiments, the armrest 1300 may include additional components such as a speaker, a microphone, a joystick, or one or more integrated sensors such as a heart rate sensor or a temperature sensor. In some examples, the armrest 1300 may also include convenience components such as a cupholder.

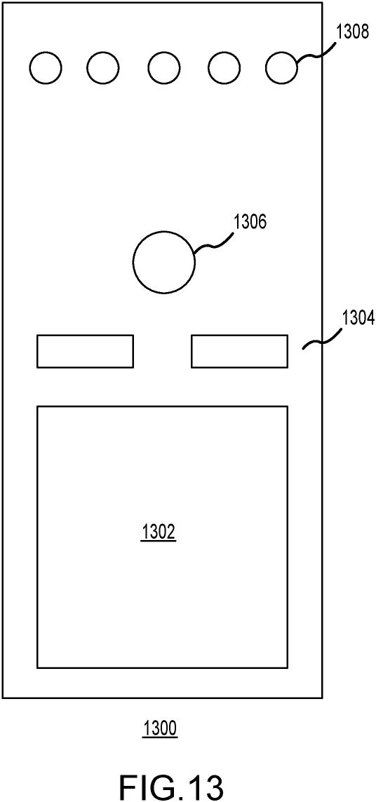

FIG. 14 is a depiction of a cushion 1400 including multiple inflatable portions in accordance with some embodiments of the disclosed subject matter.

Cushion 1400 may include a plurality of inflatable chambers. For example, cushion 1400 may include a plurality of inflatable chambers formed as concentric circles or ovals, such as inflatable chambers 1402, 1404, and 1406. Cushion 1400 may also include one or more chambers of a different size or shape, such as inflatable chamber 1408. Each of the inflatable chambers 1402, 1404, 1406, and 1408 shown in FIG. 14 may be divided into two or more inflatable chambers. For examples, the cushion 1400 may include inflatable chambers 1408a, 1408b, 1408c, and 1408d as indicated by the dashed lines. Each of the inflatable chambers may include one or more chamber pressure sensors located inside the chamber or at an opening thereof. In some other examples, the cushion 1400 may include a plurality of square, rectangular, circular, or other regularly shaped chambers. In some examples, the cushion may include more or smaller cushions around areas where finer adjustments may be needed, such as at the back and toward the middle of the cushion 1400.

The cushion 1400 may also include one or more surface pressure sensors located on the surface of the cushion. In some examples, the cushion 1400 may include one or more surface pressure sensors located above each inflatable chamber. In some examples, the surface pressure sensors may be located at regular distance intervals (e.g., every square inch) on the cushion 1400. In some other examples, the surface pressure sensors may be clustered around locations where a user is likely to experience pressure or pain. For example, the cushion 1400 may have more surface pressure sensors located near the middle and the back of the cushion 1400.

In some embodiments, an ActivTrak feature that actively tracks a wheelchair wherever it may be using GPS and mesh networks in tandem with pressure sensors to ensure the patient is in the chair. ActivTrak may allow one or more persons to actively track a seating system wherever it may be, for example, by using a GPS or other location tracker (such as location tracker 412) built into the chair. A person may use a remote computing system to request and receive information about the location of the tracker through a communication system such as a mesh network. The location tracker may work in tandem with one or more pressure sensors to ensure that the patient is still in the seating system.

In some embodiments, a FallSense feature may actively monitor if the patient has fallen from the chair, through algorithms and pressure sensors in both seat and back. In some examples, a DirectRecord feature may automatically send all biometric data to an electronic medical records system for use in telemedicine applications and can assist in developing specific treatments for the users. The ability to send medical data directly to a medical provider during a telemedicine appointment may make such appointments more effective because the medical provider can rely on sensor data rather than qualitative input provided by the patient.

In some embodiments, a SkinGuard feature may use moisture sensors and pressure sensors to identify areas of rise in the development of bed sores, skin tear, or infections. The ability to identify areas of potential danger provides may provide substantial improvements in health outcomes for wheelchair users because the change of skin damage is higher for people who spend most of their time in a seated or prone position such as when using a wheelchair. Wheelchair users frequently develop rashes and sores, particularly pressure injuries. And skin can also be damaged by sweat, feces, or urine, making pressure injuries more likely and harder to heal. SkinGuard may provide an early warning of potential danger areas so skin damage can be avoided or quickly addressed.

In some embodiments, a ComfortFlow feature may leverage SkinGuard technology to automatically adjust the cushions to reduce pressure through inputs from the user or automatically through specific algorithms, commands, and prompts.

The disclosed subject matter includes, but is not limited to, the following embodiments, and variations of these that would occur to those of skill in the art upon review of the present disclosure:

Embodiment 1. A seating system comprising:
one or more processors;
one or more sensors;
one or more memory units for storing readings from the sensors; and
one or more communication systems for transmitting the readings from the biometric sensors to a remote computer system.

Embodiment 2. The seating system of Embodiment 1, wherein the seating system is a wheelchair.

Embodiment 3. The seating system of Embodiments 1 or 2, wherein the one or more sensors includes at least one of a weight sensor, a temperature sensor, a pressure sensor, and a blood oxygen sensor.

Embodiment 4. The seating system of any of Embodiments 1 through 3, wherein the one or more communication systems include at least one of a Bluetooth communication system and a Wi-Fi communication system.

Embodiment 5. The seating system of any of Embodiments 1 through 4, further including a location tracker.

Embodiment 6. The seating system of any of Embodiments 1 through 6, further including a sensor for measuring the speed of the seating system.

Embodiment 7. The seating system of any of Embodiments 1 through 6, further including one or more indicators.

Embodiment 8. The seating system of Embodiment 7, wherein the processor is configured to active at least one of the one or more indicators upon detecting an unsafe condition.

Embodiment 9. The seating system of Embodiment 8, wherein the processor displays a message on a display screen when the unsafe condition is detected.

Embodiment 10. The seating system of any of Embodiments 1 through 9, further comprising a seat portion having a seating surface and a back portion having a support surface.

Embodiment 11. The seating system of Embodiment 10, wherein the seating surface of the seat portion is generally perpendicular to the support surface of the back portion when the seating system is configured in a seating configuration.

Embodiment 12. The seating system of Embodiment 10, wherein the seating surface of the seat portion is generally parallel to and facing a same direction as the support surface of the back portion when the seating system is configured in a recumbent configuration.

Embodiment 13. The seating system of Embodiment 10, wherein the seating surface of the seat portion is generally parallel to and facing an opposite direction as the support surface of the back portion when the seating system is configured in a stored configuration.

Embodiment 14. The seating system of any of Embodiments 10 through 13, wherein the seat portion is detachably connected to a frame of the seating system on a first lateral side thereof.

Embodiment 15. The seating system of Embodiment 14, wherein the seat portion is rotatably attached to the frame on a second lateral side thereof, the second lateral side being opposite the first lateral side, where the seat portion may be attached to the frame by any suitable rotatable connection such one or more hinges, rotary joints, pivot joints, or ball joints..

Embodiment 16. The seating system of any of Embodiments 10 through 13, wherein the seat portion is detachably connected to a frame of the seating system on a back side thereof.

Embodiment 17. The seating system of Embodiment 16, wherein the seat portion is rotatably attached to the frame on a front side thereof, the front side being opposite the back side, where the seat portion may be attached to the frame by any suitable rotatable connection such one or more hinges, rotary joints, pivot joints, or ball joints..

Embodiment 18. The seating system of any of Embodiments 10 through 17, wherein the seat portion is slidably connected to the frame of the seating system.

Embodiment 19. The seating system of any of Embodiments 10 through 17, wherein a first portion of the seat portion is slidably connected to a second portion of the seat portion.

Embodiment 20. The seating system of any of Embodiments 10 through 19, wherein the back portion is rotatably connected to the frame along a first edge, the first edge being the edge of the back portion proximate the seat portion when the seating system is configured in a seating configuration, where the back portion may be attached to the frame by any suitable rotatable connection such one or more hinges, rotary joints, pivot joints, or ball joints..

Embodiment 21. The seating system of any of Embodiments 1 through 20, further including a first primary wheel having a first hub and a second primary wheel having a second hub.

Embodiment 22. The seating system of Embodiment 21, further comprising an extendable axle, the extendable axle being one of (1) a single extendable axle having a linear or curved shape connecting the first hub directly to the second hub or (2) a first extendable axle portion connecting the first hub to a frame of the seating system and a second extendable axle portion connecting the second hub to the frame, at least one of the first extendable axle portion and the second extendable axle portion being capable of being shortened to decrease the distance between the first primary wheel and the second primary wheel.

Embodiment 23. The seating system of any of Embodiments 21 or 22, wherein the first primary wheel has a single bar, the bar having a first spoke connecting the first hub to a first position on a rim of the first primary wheel and a second spoke connecting the first hub to a second location on the rim of the first primary wheel, the second location being located radially opposite the first location.

Embodiment 24. The seating system of any of Embodiments 21 through 23, further including a braking mechanism comprising (1) a brake and (2) a brake control connected to the brake, wherein the brake control is located radially outside of the first primary wheel and a second primary wheel.

Embodiment 25. The seating system of Embodiment 24, wherein the brake control is disposed on the frame of the seating system on a same side as the first primary wheel and at a distance from the first primary wheel.

Embodiment 26. The seating system of Embodiment 25, wherein the distance between the brake control and the first primary wheel is adjustable.

Embodiment 27. The seating system of Embodiments 24 through 26, wherein the brake control configured to activate the brake by pushing or pulling the brake control towards the frame.

Embodiment 28. The seating system of Embodiments 24 through 27, wherein the brake control is connected to the brake by one of a hydraulic connection, a pneumatic connection, or an electrical connection.

Embodiment 29. The seating system of Embodiments 24 through 28, wherein the brake control comprises a lever which may be a hydraulic or pneumatic control lever.

Embodiment 30. The seating system of any of Embodiments 1 through 29, further including one or more armrests.

Embodiment 31. The seating system of Embodiment 30, wherein one or more indicator lights associated with the one or more sensors are located on the one or more armrests.

Embodiment 32. The seating system of Embodiment 31, wherein the indicator lights include a first state associated with a safe condition and a second state associated with an emergency condition.

Embodiment 33. The seating system of Embodiment 32, wherein the indicator lights include a third state associated with a warning condition.

Embodiment 34. The seating system of Embodiment 33, wherein each state is associated with a range of values for the corresponding sensor.

Embodiment 35. The seating system of any of Embodiments 30 through 34, wherein the armrests are rotatable.

Embodiment 36. The seating system of any of Embodiments 30 through 35, wherein the armrests include a removable portion.

Embodiment 37. The seating system of any of Embodiments 10 through 36, further including one or more footpads.

Embodiment 38. The seating system of Embodiment 37, wherein the one or more footpads are connected to a frame of the seating system by one or more support rods.

Embodiment 39. The seating system of Embodiment 38, wherein the support rods are capable of being extended or retracted to adjust the height of the footpads.

Embodiment 40. The seating system of Embodiments 38 or 39, wherein the support rods are rotatably coupled to the frame.

Embodiment 41. The seating system of Embodiment 40, wherein the support rods extend in a first direction generally perpendicular to the seat portion when the seating system is in a seating configuration and extend in a second direct generally parallel to the seat portion when the seating system is in a recumbent configuration.

Embodiment 42. The seating system of any of Embodiments 1 through 41, further including a cushion.

Embodiment 43. The seating system of Embodiment 42, wherein the cushion includes a plurality of inflatable chambers.

Embodiment 44. The seating system of Embodiments 42 or 43, further including a fluid source coupled to the cushion.

Embodiment 45. The seating system of Embodiment 44, wherein the fluid source is a gas source.

Embodiment 46. The seating system of Embodiment 45, wherein the fluid source is an air source.

Embodiment 47. The seating system of Embodiments 44 through 46, wherein the fluid source includes a refillable or replaceable tank.

Embodiment 48. The seating system of Embodiments 44 through 46, wherein the fluid source includes a compressor.

Embodiment 49. The seating system of any of Embodiments 43 through 48, wherein a processor is configured to selectively inflate or deflate the plurality of inflatable chambers.

Embodiment 50. The seating system of Embodiment 49, wherein the processor is configured to selectively inflate or deflate the plurality of inflatable chambers based on instructions stored in memory.

Embodiment 51. The seating system of Embodiments 49 or 50, wherein the processor is configured to selectively inflate or deflate based on user input.

Embodiment 52. The seating system of Embodiments 43 through 51, wherein the cushion includes one or more surface pressure sensors.

Embodiment 53. The seating system of Embodiment 52, wherein a processor is configured to selectively inflate or deflate chambers of the cushion based on pressure readings from the one or more pressure sensors.

Embodiment 54. The seating system of Embodiments 43 through 53, wherein the cushion includes one or more in-chamber pressure sensors.

Embodiment 55. The seating system of Embodiment 54, wherein a processor is configured to selectively inflate or deflate chambers of the cushion based on pressure readings from the one or more in-chamber sensors.

Embodiment 56. The seating system of Embodiments 43 through 55, further including at least one valve.

Embodiment 57. The seating system of Embodiment 56, wherein at least one valve is printed on or otherwise integrally formed with at least one of the inflatable chambers.

Embodiment 58. The seating system of Embodiments 56 or 57, wherein the at least one valve includes an intake valve located between the fluid source and each inflatable chamber.

Embodiment 59. The seating system of Embodiment 56 through 58, wherein the at least one valve includes an exhaust valve.

Embodiment 60. The seating system of Embodiments 56 through 59, wherein the at least one valve includes a combination intake valve and exhaust valve.

Embodiment 61. The seating system of Embodiments 56 through 60, wherein the at least one valve valve includes a first surface positioned perpendicular to the direction of fluid flow when the valve is closed and second and third surfaces angled relative to the first surface and configured to engage first and second angled surfaces of a valve seat when the valve is closed.

Embodiment 62. The seating system of Embodiments 10 through 61, further including a headrest.

Embodiment 63. The seating system of Embodiments 10 through 62, further including one or more cupholders.

Embodiment 64. A system comprising:
a seating system as described in any of Embodiments 1 through 56; and
one or more remote computers for receiving and storing sensor data from the one or more sensors of the seating system.

Embodiment 65. The system of Embodiment 64, wherein the seating system is configured to transmit the sensor data to the one or more remote computers.

Embodiment 66. The system of Embodiments 64 or 65, wherein the one or more remote computers are configured to detect a warning condition based on the sensor data from the one or more sensors.

Embodiment 67. The system of Embodiment 66, wherein the one or more remote computers are further configured to transmit an alert to at least one of the user, the caregiver, the medical provider, or another authorized person when a warning condition is detected.

Embodiment 68. The system of any of Embodiments 64 through 67, wherein the one or more remote computers are further configured to receive a request for access to the sensor data, determine whether the requestor is authorized to access the sensor data based on authorization data received from the user, and transmit the sensor data to the requestor when the requestor is determined to be an authorized party.

Embodiment 69. The system of any of Embodiments 1 through 68, further comprising controlling one or more features of the seating system based on the sensor data.

Embodiment 70. The system of any of Embodiments 1 through 69, wherein the system is compliant with applicable regulations such as HIPAA and/or GDPR and/or wherein access to the sensor data through a requesting device is managed in compliance with such applicable regulations.

Embodiment 71. A method for collapsing a seating system as described in Embodiments 1 through 70 from a seating configuration to a stored configuration, comprising (1) rotating a back portion of the seating system from a first position generally perpendicular to a seat portion of the seating system to a second position generally parallel with the seat portion, wherein the support surface of the back portion is located proximate the seat surface of the seat portion after the back portion is rotated; (2) rotating the seat portion of the seating system; and (3) shortening the length of an extendable axle to move a first primary wheel towards a second primary wheel.

Embodiment 72. The method of Embodiment 71, further comprising detaching the seat portion from the frame of the seating system on a first side thereof and rotating the seat portion about the frame on a second side thereof opposite the first side.

Embodiment 73. The method of Embodiment 72, wherein the first side is a first lateral side of the seat portion and the second side is a second lateral side of the seat portion.

Embodiment 74. The method of Embodiment 73, wherein the seat portion and the back portion are located generally parallel with and between the primary wheels after the seat portion is rotated.

Embodiment 75. The method of Embodiment 72, wherein the first side is a back side of the seat portion and the second side is a front side of the seat portion.

Embodiment 76. The method of Embodiment 75, wherein the seat portion and the back portion are located above the primary wheels after the seat portion is rotated and the length of the extendable axle is shortened.

Embodiment 77. The method of Embodiments 71 through 76, further comprising axially rotating at least one push bar.

Embodiment 78. The method of Embodiments 71 through 77, further comprising rotating support rods between a first position in which the support rods extend generally downward from the seat portion and a second position in which the support rods extend generally parallel with and towards the seat portion.

Embodiment 79. The method of Embodiments 71 through 78, further comprising rotating foot pads between a first position generally perpendicular to support rods and a second position generally parallel with the support rods.

Embodiment 80. The method of Embodiments 71 through 79, further comprising rotating one or more calf pads between a first position and a second position generally perpendicular to the first position, wherein the calf pads are generally parallel with the plane of the primary wheels when in the second position.

Embodiment 81. A method for adjusting a seating system between a seating configuration and a recumbent configuration, comprising (1) rotating a back portion from a first position generally perpendicular with a seat portion to a second position generally parallel with the seat portion, with the seating surface of the seat portion and the support surface of the back portion facing the same direction; and (2) rotating support rods from a first position in which the support rods extend generally downward from the seat portion to a second position in which the support rods extend generally parallel with and away from the seat portion.

Embodiment 82. The method of Embodiment 81, further comprising axially rotating at least one push bar.

Embodiment 83. A seating system comprising a single-piece seating portion, a first primary wheel located on a first lateral side of the seating portion, and a second primary wheel located on a second lateral side of the seating portion opposite the first lateral side.

Embodiment 84. The seating system of Embodiment 83, wherein the single-piece seating portion comprises plastic woven mesh.

Embodiment 85. The seating system of Embodiments 83 or 84, further comprising a frame connected to the single-piece seating portion.

Embodiment 86. The seating system of Embodiment 85, wherein the frame comprises lightweight metals such as aluminum or titanium.

Embodiment 87. The seating system of Embodiments 85 or 86, wherein a hub of the first primary wheel is connected directly to the frame.

Embodiment 88. The seating system of Embodiments 83 through 87, wherein the first primary wheel comprises a single bar comprising a first spoke connecting the hub of the first primary wheel to a first location on the rim of the first primary wheel and a second spoke connecting the hub of the first primary wheel to a second location on the rim of the first primary wheel opposite the first location.

Embodiment 89. The seating system of Embodiments 83 through 88, wherein the wheel comprises lightweight metals such as aluminum or titanium.

Embodiment 90. The seating system of Embodiments 83 through 89, further comprising a circular brake located laterally outside of the rim of the first primary wheel.

Embodiment 91. The seating system of Embodiments 83 through 90, further comprising first and second secondary wheels coupled to the frame.

Embodiment 92. The seating system of Embodiments 83 through 91, further comprising a support rod extending downward from the frame.

Embodiment 93. The seating system of Embodiment 92, wherein the support rod comprises first and second telescoping rod portions.

Embodiment 94. The seating system of Embodiments 92 or 93, further comprising a footpad connected to the frame via the support rod.

Embodiment 95. The seating system of Embodiments 92 through 94, wherein the support rod and/or footpad comprises lightweight metals such as aluminum or titanium.

Embodiment 96. The seating system of Embodiments 92 through 95, further comprising a calf pad attached to the support rod.

Embodiment 97. The seating system of Embodiment 96, wherein the calf pad comprises plastic woven mesh.

Embodiment 98. The seating system of Embodiments 83 through 97, further comprising a cushion located on the single-piece seating portion.

Embodiment 99. The seating system of Embodiment 98, wherein the cushion comprises at least one of a pressure sensor, a moisture sensor, a heart rate sensor, and a temperature sensor.

Embodiment 100. The seating system of Embodiments 98 or 99, wherein the cushion is configured as described in Embodiments 43 through 61.

Embodiment 101. The seating system of Embodiments 83 through 100, wherein the seating system is a wheelchair.

Embodiment 102. The seating system of Embodiments 83 through 101, further comprising electronic components as described in Embodiments 4 through 9.

Embodiment 103. The seating system of Embodiments 99 or 102, further comprising one or more remote computers for receiving and storing sensor data as described in Embodiments 64 through 70.

Embodiment 104. The seating system of Embodiments 1 through 70 or 83 through 103, further comprising a photovoltaic cell.

Embodiment 105. The seating system of Embodiments 1 through 70 or 83 through 104, further comprising at least one reflector located on the seating system.

Embodiment 106. The seating system of Embodiments 1 through 70 or 83 through 105, further comprising at least one locking mechanism configured to lock at least one rotatable, detachable, or slidable component of the seating system during use.

Embodiment 107. A seating system comprising:
a first primary wheel comprising:
   a first rim;
   a first hub; and
   a single bar consisting of a first spoke connecting the first hub to a first location on the first rim and a second spoke connecting the first hub to the first rim at a second location radially opposite the first location, wherein the first and second spokes have a width of at least one inch;
a second primary wheel located laterally opposite the first primary wheel;
an extendable axle connecting the first primary wheel to the second primary wheel, the extendable axle comprising at least two telescoping portions;
a support frame comprising:
   a first frame portion on a first lateral side of the seating system; and
   a second frame portion on a second lateral side of the seating system opposite the first lateral side, wherein the first frame portion is connected to
   the second frame portion via the extendable axle;
a braking mechanism comprising:
   a brake located laterally outside of the first primary wheel; and
   a brake control disposed on the first frame portion at a first distance from the first primary wheel, the brake control comprising an outer surface, wherein the brake control causes the brake to activate when the outer surface is moved towards the first frame portion, wherein the first distance is adjustable and wherein the brake control comprises at least one of a hydraulic brake control and a pneumatic brake control.
a seat portion comprising a seating surface, the seat portion being detachably connected to the first frame portion and rotatably connected to the second frame portion, and wherein the seat portion is slidable relative to the frame;
a back portion comprising a support surface, the back portion being rotatably connected to the seat portion;
at least one push bar rotatably connected to the extendable axle;
at least one armrest rotatably connected to the extendable axle;
at least one support rod rotatably connected to the frame, the at least one support rod comprising at least two telescoping rods;
at least one footpad rotatably connected to the at least one support rod; and an inflatable cushion located on the seat portion, the inflatable cushion comprising:
   two or more pressure sensors, the two or more pressure sensors comprising at least one surface pressure sensor and at least one chamber pressure sensor;
   one or more thin film moisture sensors; and
   a plurality of inflatable chambers, each of the inflatable chambers being connected to a fluid source by at least one valve controlled by a control circuit, wherein the control circuit is programmed to adjust a pressure in each of the inflatable chambers based on measurements by the two or more pressure sensors, and wherein at least a portion of the at least one valve is printed on and integral with the inflatable chamber,
wherein the seating system is configurable in a seating configuration, a recumbent configuration, and a stored configuration, wherein:
   the seat portion is generally perpendicular to the back portion, the extendable axle has a first length, the at least one support rod extends generally downward, and the at least one footpad is generally perpendicular to the at least one support rod when the seating system is configured in the seating configuration;
   the seat portion is generally parallel with the back portion, the seating surface of the seat portion faces in a same direction as the support surface of the back portion, and the at least one support rod extends generally parallel with and away from the seat portion when the seating system is configured in the recumbent configuration; and
   the seating surface of the seat portion faces towards the support surface of the back portion, the extendable axle has a second length less than the first length, the at least one footpad is generally parallel with the at least one support rod, and the seat portion and the back portion are located between the first primary wheel and the second primary wheel when the seating system is configured in the stored configuration.

Embodiment 108. A seating system comprising:
a first primary wheel comprising:
   a first rim;
   a first hub; and
   a single bar consisting of a first spoke connecting the first hub to a first location on the first rim and a second spoke connecting the first hub to the first rim at a second location radially opposite the first location, wherein the first and second spokes have a width of at least one inch;
a second primary wheel located laterally opposite the first primary wheel;
an extendable axle connecting the first primary wheel to the second primary wheel, the extendable axle comprising at least two telescoping portions;
a support frame comprising:
   a first frame portion on a first lateral side of the seating system; and
   a second frame portion on a second lateral side of the seating system opposite the first lateral side, wherein the first frame portion is connected to the second frame portion via the extendable axle;
a braking mechanism comprising:
   a brake located laterally outside of the first primary wheel; and
   a brake control disposed on the first frame portion at a first distance from the first primary wheel, the brake control comprising an outer surface, wherein the brake control causes the brake to activate when the outer surface is moved towards the first frame portion, wherein the first distance is adjustable and wherein the brake control comprises at least one of a hydraulic brake control and a pneumatic brake control.
a seat portion comprising a seating surface, a front edge, and a back edge opposite the front edge, the seat portion being rotatably connected to each of the first frame portion and the second frame portion, and wherein the seat portion is slidable relative to the frame;
the back portion comprising a support surface, the back portion being rotatably connected to the seat portion;
at least one push bar rotatably connected to the extendable axle;
at least one armrest rotatably connected to the extendable axle;
at least one support rod rotatably connected to the frame, the at least one support rod comprising at least two telescoping rods;
at least one footpad rotatably connected to the at least one support rod; and an inflatable cushion located on the seat portion, the inflatable cushion comprising:
   two or more pressure sensors, the two or more pressure sensors comprising at least one surface pressure sensor and at least one chamber pressure sensor;
   one or more thin film moisture sensors; and
   a plurality of inflatable chambers, each of the inflatable chambers being connected to a fluid source by at least one valve controlled by a control circuit, wherein the control circuit is programmed to adjust a pressure in each of the inflatable chambers based on measurements by the two or more pressure sensors, and wherein the at least a portion of the at least one valve is printed on and integral with the inflatable chamber,
wherein the seating system is configurable in a seating configuration, a recumbent configuration, and a stored configuration, wherein:
   the seat portion is generally perpendicular to the back portion, the back edge of the seat portion is proximate the back portion, the extendable axle has a first length, the at least one support rod extends generally downward, and the at least one footpad is generally perpendicular to the at least one support rod when the seating system is configured in the seating configuration;
   the seat portion is generally parallel with the back portion, the seating surface of the seat portion faces in a same direction as the support surface of the back portion, and the at least one support rod extends generally parallel with and away from the seat portion when the seating system is configured in the recumbent configuration; and
   the seating surface of the seat portion faces towards the support surface of the back portion, the extendable axle has a second length less than the first length, the at least one footpad is generally parallel with the at least one support rod, the back edge of the seat portion is elevated relative to the front edge of the seat portion, and the seat portion and the back portion are not located between the first primary wheel and the second primary wheel when the seating system is configured in the stored configuration. Embodiment 109. The seating system of Embodiments 1 through 108, wherein the seating system is a mobility system.

Embodiment 110. The seating system of Embodiments 1 through 108, wherein the seating system is a mobile seating device.

Embodiment 111. The seating system of Embodiments 1 through 108, wherein the seating system is a human transport device.

The foregoing merely illustrates the principles of the disclosed subject matter. Various modifications and alterations to the described embodiments will be apparent to those skilled in the art in view of the inventors’ teachings herein. It will thus be appreciated that those skilled in the art will be able to devise numerous systems and methods which, although not explicitly shown or described herein, embody the principles of the disclosed subject matter and thus are within it spirit and scope. Such modifications and alterations are within the scope and spirit of the present invention, as set forth in the following claims. Further, the invention(s) described herein is capable of other embodiments and of being practiced or of being carried out in various ways. It is to be understood that the phraseology and terminology used herein is for the purpose of description and should not be regarded as limiting.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations or aspects other than those discussed above. This method of disclosure is not to be interpreted as reflecting an intention that the claimed disclosure requires more features than are expressly recited in each claim. Rather, as the foregoing claims reflect, inventive aspects lie in less than all features of a single foregoing disclosed embodiment, configuration, or aspect. Thus, the following claims are hereby incorporated into this Detailed Description, which each claim standing on its own as a separate preferred embodiment of the disclosure.

The features of the various embodiments described herein are not intended to be mutually exclusive when the nature of those features does not require mutual exclusivity. Instead, the features and aspects of one embodiment may be combined with features or aspects of another embodiment. Additionally, the description of a particular element with respect to one embodiment may apply to the use of that particular element in another embodiment, regardless of whether the description is repeated in connection with the use of the particular element in another embodiment.

Embodiments and examples provided herein are intended to be illustrative and non-limiting. Thus, any example or set of examples provided to illustrate one or more aspects of the present disclosure should not be considered to comprise the entire set of possible embodiments of the aspect in question. Examples may be identified by use of the terms or phrases “for example,” “such as,” “by way of example,” “e.g.,” and other language commonly understood to indicate that what follows is an example.

What is claimed is:

1. A seating system comprising:
a seat portion having a seating surface; and
a back portion having a support surface, the back portion being rotatable relative to the seat portion,
wherein the seating system is configurable in a seating configuration in which the seating surface of the seat portion is generally perpendicular to the support surface of the back portion, and where the seating system is also configurable in a stored configuration in which the seating surface of the seat faces towards the support surface of the back portion.

2. The seating system of claim 1, wherein the seat portion is slidable relative to a frame of the seating system.

3. The seating system of claim 1, wherein the seat portion is detachably connected to a frame of the seating system on a first lateral side of the seat portion and wherein the seat portion is rotatably connected to the frame on a second lateral side of the seat portion opposite the first lateral side.

4. The seating system of claim 1, further comprising a first primary wheel having a first hub and a second primary wheel having a second hub.

5. The seating system of claim 4, wherein the first hub is connected to the second hub via an extendable axle.

6. The seating system of claim 5, wherein the extendable axle is configurable in an elongated configuration when the seating system is configured in the seating configuration and wherein the extendable axle is configurable in a shortened configuration when the seating system is configured in the stored configuration.

7. The seating system of claim 6, wherein the first primary wheel and the second primary wheel are generally parallel with the seating surface of the seat portion and the support surface of the back portion when the seating system is in the stored configuration.

8. The seating system of claim 4, further comprising a braking mechanism comprising a brake configured to brake the first primary wheel when activated by a brake control, wherein the brake control is located radially outside of the first primary wheel and the second primary wheel.

9. The seating system of claim 8, wherein the brake control is disposed on a frame of the seating system on a same side as the first primary wheel and a distance between the brake control and the first primary wheel is adjustable.

10. The seating system of claim 1, further comprising a footpad connected to the frame by an adjustable support rod.

11. The seating system of claim 10, further comprising at least one calf pad connected to the adjustable support rod.

12. The seating system of claim 10, wherein the footpad is configurable in a first position generally perpendicular to the adjustable support rod and wherein the footpad is configurable in a second position generally parallel to the adjustable support rod.

13. The seating system of claim 10, wherein the adjustable support rod is rotatably connected to the frame, wherein the support rod is configured in a first position in which the support rod extends downward from the frame when the seating system is in the seating configuration, and wherein the support rod is configured in a second position in which the support rod is generally parallel with and located underneath the seat portion when the seating system is in the stored configuration.

14. The seating system of claim 1, wherein the seating system is further configurable in a recumbent position in which the seating surface of the seat portion is generally parallel to and facing a same direction as the support surface of the back portion.

15. The seating system of claim 14, further comprising an adjustable support rod and a footpad connected to the adjustable support rod, wherein the adjustable support rod extends downward a frame of the seating system when the seating system is in the seating configuration and wherein the adjustable support rod extends generally parallel with and away from the seat portion when the seating system is in the recumbent configuration.

16. The seating system of claim 15, further comprising a control mechanism that moves the adjustable support rod, the control mechanism being at least one of an electrical control mechanism or a mechanical control mechanism.

17. The seating system of claim 1, further comprising an armrest comprising an attachment portion and a removable portion that is removably attached to the attachment portion.

18. The seating system of claim 1, further comprising an inflatable cushion located on the seating surface of the seat portion, wherein the inflatable cushion comprises a plurality of inflatable chambers connected to a fluid source via an intake valve.

19. The seating system of claim 18, wherein the intake valve comprises a poppet connected to an actuator, the poppet comprising:
a first surface perpendicular to a direction of fluid flow through a conduit connecting the fluid source to the intake valve; and
second and third surfaces angled with respect to the first surface and configured to engage with first and second angled surfaces of a valve seat when the intake valve is closed.

20. A seating system comprising:
a first primary wheel comprising:
a first rim;
a first hub; and
a single bar consisting of a first spoke connecting the first hub to a first location on the first rim and a second spoke connecting the first hub to the first rim at a second location radially opposite the first location, wherein the first and second spokes have a width of at least one inch;
a second primary wheel located laterally opposite the first primary wheel;
an extendable axle connecting the first primary wheel to the second primary wheel,
the extendable axle comprising at least two telescoping portions;
a support frame comprising:
a first frame portion on a first lateral side of the seating system; and
a second frame portion on a second lateral side of the seating system opposite the first lateral side, wherein the first frame portion is connected to the second frame portion via the extendable axle;
a braking mechanism comprising:
a brake located laterally outside of the first primary wheel; and
a brake control disposed on the first frame portion at a first distance from the first primary wheel, the brake control comprising an outer surface,
wherein the brake control causes the brake to activate when the outer surface is moved towards the first frame portion, wherein the first distance is adjustable and wherein the brake control comprises at least one of a hydraulic brake control and a pneumatic brake control;
a seat portion comprising a seating surface, the seat portion being detachably connected to the first frame portion and rotatably connected to the second frame portion, and wherein the seat portion is slidable relative to the frame;
a back portion comprising a support surface, the back portion being rotatably connected to the seat portion;
at least one push bar rotatably connected to the extendable axle;
at least one armrest rotatably connected to the extendable axle;
at least one support rod rotatably connected to the frame, the at least one support rod comprising at least two telescoping rods;
at least one footpad rotatably connected to the at least one support rod; and
an inflatable cushion located on the seat portion, the inflatable cushion comprising:
two or more pressure sensors, the two or more pressure sensors comprising
at least one surface pressure sensor and at least one chamber pressure sensor;
one or more thin film moisture sensors; and
a plurality of inflatable chambers, each of the inflatable chambers being connected to a fluid source by at least one valve controlled by a control circuit, wherein the control circuit is programmed to adjust a pressure in each of the inflatable chambers based on measurements by the two or more pressure sensors, and wherein at least a portion of the at least one valve is printed on and integral with the inflatable chamber,
wherein the seating system is configurable in a seating configuration, a recumbent configuration, and a stored configuration, wherein:
the seat portion is generally perpendicular to the back portion, the extendable axle has a first length, the at least one support rod extends generally downward, and the at least one footpad is generally perpendicular to the at least one support rod when the seating system is configured in the seating configuration;
the seat portion is generally parallel with the back portion, the seating surface of the seat portion faces in a same direction as the support surface of the back portion, and the at least one support rod extends generally parallel with and away from the seat portion when the seating system is configured in the recumbent configuration; and
the seating surface of the seat portion faces towards the support surface of the back portion, the extendable axle has a second length less than the first length, the at least one footpad is generally parallel with the at least one support rod, and the seat portion and the back portion are located between the first primary wheel and the second primary wheel when the seating system is configured in the stored configuration.

21. A seating system comprising:
a first primary wheel comprising:
a first rim;
a first hub; and
a single bar consisting of a first spoke connecting the first hub to a first location on the first rim and a second spoke connecting the first hub to the first rim at a second location radially opposite the first location, wherein the first and second spokes have a width of at least one inch;
a second primary wheel located laterally opposite the first primary wheel;
an extendable axle connecting the first primary wheel to the second primary wheel,
the extendable axle comprising at least two telescoping portions;
a support frame comprising:
a first frame portion on a first lateral side of the seating system; and
a second frame portion on a second lateral side of the seating system opposite the first lateral side, wherein the first frame portion is connected to the second frame portion via the extendable axle;
a braking mechanism comprising:
a brake located laterally outside of the first primary wheel; and
a brake control disposed on the first frame portion at a first distance from the first primary wheel, the brake control comprising an outer surface,
wherein the brake control causes the brake to activate when the outer surface is moved towards the first frame portion, wherein the first distance is adjustable and wherein the brake control comprises at least one of a hydraulic brake control and a pneumatic brake control;
a seat portion comprising a seating surface, a front edge, and a back edge opposite the front edge, the seat portion being rotatably connected to each of the first frame portion and the second frame portion, and wherein the seat portion is slidable relative to the frame;
the back portion comprising a support surface, the back portion being rotatably connected to the seat portion;
at least one push bar rotatably connected to the extendable axle;
at least one armrest rotatably connected to the extendable axle;
at least one support rod rotatably connected to the frame, the at least one support rod comprising at least two telescoping rods;
at least one footpad rotatably connected to the at least one support rod; and
an inflatable cushion located on the seat portion, the inflatable cushion comprising:
two or more pressure sensors, the two or more pressure sensors comprising
at least one surface pressure sensor and at least one chamber pressure sensor;
one or more thin film moisture sensors; and
a plurality of inflatable chambers, each of the inflatable chambers being connected to a fluid source by at least one valve controlled by a control circuit, wherein the control circuit is programmed to adjust a pressure in each of the inflatable chambers based on measurements by the two or more pressure sensors, and wherein the at least a portion of the at least one valve is printed on and integral with the inflatable chamber,
wherein the seating system is configurable in a seating configuration, a recumbent configuration, and a stored configuration, wherein:
the seat portion is generally perpendicular to the back portion, the back edge of the seat portion is proximate the back portion, the extendable axle has a first length, the at least one support rod extends generally downward, and the at least one footpad is generally perpendicular to the at least one support rod when the seating system is configured in the seating configuration;
the seat portion is generally parallel with the back portion, the seating surface of the seat portion faces in a same direction as the support surface of the back portion, and the at least one support rod extends generally parallel with and away from the seat portion when the seating system is configured in the recumbent configuration; and
the seating surface of the seat portion faces towards the support surface of the back portion, the extendable axle has a second length less than the first length, the at least one footpad is generally parallel with the at least one support rod, the back edge of the seat portion is elevated relative to the front edge of the seat portion, and the seat portion and the back portion are not located between the first primary wheel and the second primary wheel when the seating system is configured in the stored configuration.
